(19) United States
(12) Patent Application Publication
(10) Pub. No.: US 2026/0216154 A1
(43) Pub. Date: Jul. 30, 2026

(54) POTASSIUM CHANNEL BLOCKERS OR DERIVATIVES THEREOF FOR PREVENTING, ALLEVIATING, AND/OR TREATING MORBIDITY AND MORTALITY INDUCED BY PATHOGENS OR OTHER INDUCERS OF IMMUNE EXHAUSTION AND/OR EXCESS IMMUNE ACTIVATI

(71) Applicant: University of Rochester, Rochester, NY (US)

(72) Inventors: Mark Noble, Rochester, NY (US); Margot Mayer-Proschel, Pittsford, NY (US); Maleelo Shamambo, Rochester, NY (US); Michael A. O'Reilly, Penfield, NY (US)

(21) Appl. No.: 19/146,786

(22) PCT Filed: Jan. 12, 2024
(86) PCT No.: PCT/US2024/011428

§ 371 Date: Jul. 9, 2025

Related U.S. Application Data
(60) Provisional application No. 63/479,858, filed on Jan. 13, 2023.

(51) Int. Cl. A61K 31/4409; A61K 31/19; A61K 31/197; A61K 31/40; A61K 31/4015; A61K 31/4166; A61K 31/421; A61K 31/513; A61K 31/515; A61K 31/7004; A61K 33/00; A61K 45/06; A61P 3/00; A61P 21/00

(52) U.S. Cl. CPC A61K 31/4409; A61K 31/19; A61K 31/197; A61K 31/40; A61K 31/4015; A61K 31/4166; A61K 31/421; A61K 31/513; A61K 31/515; A61K 31/7004; A61K 33/00; A61K 45/06; A61P 3/00; A61P 21/00

(57) ABSTRACT

In one aspect, the present invention is directed to compositions and methods of preventing death and severe morbidity induced by a pathogen. In another aspect, the present invention is directed to compositions and methods of preventing, alleviating, or treating an excessive immune activation (e.g., hypercytokinemia, also known as a cytokine storm and as cytokine release syndrome) induced by a pathogen or caused by other means than pathogen infection, immune system exhaustion induced by a pathogen, and/or a tissue damage or dysfunction caused by exposure to a pathogen. In another aspect, the present invention is directed to compositions and methods of preventing, alleviating, or treating the effects of pathogen infection in individuals with a compromised immune system. In some embodiments, the composition comprises at least one potassium channel blocker (e.g., 4-aminopyridine). In some embodiments, the composition can be administered with an additional therapeutic agent, such as an anticonvulsant.

### CROSS-REFERENCE TO RELATED APPLICATIONS

This application claims priority to and the benefit of U.S. Provisional Application No. 63/479,858 filed Jan. 13, 2023, the disclosure of which is incorporated herein by reference in its entirety.

### BACKGROUND OF THE INVENTION

Multiple pathogens are capable of causing death or severe morbidity after infection, with such outcomes occurring within several days to several weeks post-infection. The complexity and severity of such infections are such that it has been difficult to develop treatments for these important medical challenges.

Looking at the overall picture of pathogen infections that cause death over relatively short time periods (e.g., 3-6 weeks or less), one of the challenges to the development of treatments is that different pathogens can have differing effects on the body, the causes of death are varied, and different individuals respond to the same pathogen in different ways. On a general level, however, contributions to death may be an overly aggressive immune response to the pathogen, secondary infections with opportunistic pathogens (which can lead to still further hyper-activation of the immune response), failure of multiple organ systems and sepsis (which is itself a response to infection) and/or immune exhaustion (in which the immune system is no longer able to defeat the invading pathogen(s)). In the case of infections that impair lung function (e.g., respiratory pathogens), there may also be problems caused by insufficient oxygen transport from the lungs to the bloodstream. These challenges in lung function in turn may be caused by multiple factors, such as disruption and death of epithelial cells, damage to alveoli, vascular changes, and effects of an overly aggressive immune response.

These pathological changes represent some of the most difficult medical challenges, with limited strategies for amelioration of dysfunctions associated with, for example, infection with pathogens able to cause death or severe morbidity within a few days or weeks of infection.

One of the best studied inducers of mortality or severe morbidity is hypercytokinemia, which is also referred to as “cytokine release syndrome,” or as a “cytokine storm”. Hypercytokinemia is a potentially fatal immune reaction that involves a positive feedback loop between cytokines and immune cells, such that activated cells (and particularly activated immune cells) can release cytokines that increase the activation of immune cells, leading to still greater cytokine production and generation of a positive feedback loop associated with highly elevated levels of various cytokines in the body. Hypercytokinemia typically involves increased concentrations of cytokines, such as interferons, interleukins, chemokines, colony-stimulating factors, and tumor necrosis factors, although the precise contributors and their relative concentrations can vary with different pathogens and even with the same pathogen in different people. Such immune dysregulation can be an underlying factor in mortality resulting from many infections.

Diseases involving hypercytokinemia also often include an overproduction of immune cells and/or pro-inflammatory cytokines into the lungs of the subject, particularly in the case of respiratory pathogens. In the lungs, hypercytokinemia may result in an excessive inflammatory response. Such excessive inflammatory responses in the lung include infiltration into the lungs by immune cells and excessive secretion of proinflammatory cytokines in the lungs, which in turn attracts more immune cells into the lungs.

Septicemia, also referred to as sepsis, is a life-threatening condition caused by infection that exhibits both hypercytokinemia when the body's response to infection can damage its own organs and, paradoxically, is followed by immunodepletion and suppression of the immune system. The risk of death from sepsis is as high as 30%, for severe sepsis is as high as 50%, and when septic shock occurs mortality is as high as 80%. Sepsis affected about 49 million people in 2017, with 11 million deaths. Although septicemia is less frequent in the developed world, even here approximately 0.2 to 3 people per 1000 are affected by sepsis yearly, resulting in about a million cases per year in the United States

Pathogen infections can be asymptomatic, mild, moderate, severe, or critical. An asymptomatic form of disease does not show any symptoms in the subject. A mild form of disease may show mild forms of one or more of a wide range of symptoms, including, but not limited to, tiredness, fever, cough, breathlessness after moderate exercise, sore throat, muscle ache, headache, and diarrhea.

Mild forms of diseases may not require management of symptoms and may rely on the ability of the body to fight infections in order to achieve recovery.

A moderate form of disease may show greater levels of the same symptoms as mild disease, but differ in that they will more likely require managing the symptoms.

More severe responses to infectious pathogens differ from mild-to-moderate responses in multiple important ways that require qualitatively different kinds of medical responses. Importantly, the medical responses used in treating mild-to-moderate responses to infection generally provide little or no benefit in the case of severe or critical responses of the infected host.

Severe responses to pathogen infection may show one or more of: severe tiredness, high fever, cough, breathlessness even at rest, painful breathing, loss of appetite, loss of thirst, weight loss sore throat, muscle ache, headache, diarrhea, and confusion. Severe forms of disease typically require significant intervention for managing a potentially wide range of symptoms, including, but not limited to, pneumonia, hypoxemic respiratory failure, ARDS, sepsis, septic shock, cardiomyopathy, arrhythmia, acute kidney injury, and complications from prolonged hospitalization including secondary bacterial infections, thromboembolism, gastrointestinal bleeding, and critical illness polyneuropathy/myopathy.

In critical forms of disease caused by pathogen infection, similar symptoms may occur as in severe disease but also may be further exacerbated and require still additional types of medical intervention even beyond those used to manage severe responses to pathogen infection. Such symptoms may include, but are not limited to, severe tiredness, high fever, cough, breathlessness even at rest, painful breathing, loss of appetite, loss of thirst, sore throat, muscle ache, headache, diarrhea, confusion, severe pneumonia, ARDS, sepsis, systemic inflammatory response syndrome (SIRS), toxic shock syndrome, organ failure, coma, and death. Such critical forms of disease may require hospitalization for managing symptoms, including such outcomes as pneumonia, ARDS, sepsis, septic shock, cardiomyopathy, arrhythmia, acute kidney injury, and complications from prolonged hospitalization including secondary bacterial infections, thromboembolism, gastrointestinal bleeding, and critical illness polyneuropathy/myopathy. Ventilator assisted breathing may be required.

Diseases may also be caused by pathogen overload due to compromised immune system function and failure to control the increases in the amount of pathogen. Such compromised immune system function could be due to genetic reasons, dietary reasons, stress, aging, Alzheimer's disease, concomitant illness (such as HIV infections), cancer treatment, radiation, or multiple other reasons. Thus, the damage caused by excessive immune activation can also be associated, paradoxically, with a failure of the immune system to control the spread of the pathogen(s) in the body. In such cases, the ability of the pathogens themselves to cause tissue damage can lead to severe and even lethal consequences.

The fact that there are so many different symptoms and manifestations of disease caused by pathogen infection makes treatment even more challenging. A focus on managing individual symptoms can leave other types of damage unchecked. Attempts to develop treatments able to cause reductions in one aspect of tissue dysfunction may leave other aspects of tissue dysfunction unchecked.

Another challenge to the development of treatments for infections that are able to cause death or severe morbidity within a few days or weeks of infection is that there are many pathogens, including viruses, bacteria, and even fungal infections that are able to cause infections that are lethal over relatively short time periods (e.g., 3-4 weeks or less).

Some of the most frequent pathogens that are potentially lethal are acute respiratory infections (ARIs), which are among the leading causes of death in the world. These pathogens are introduced into the body via breathing and thus are easily communicable. ARIs are defined as infections of the respiratory system, caused by viruses or bacteria, with an evolution of less than 15 days. They most frequently manifest as cough, nasal congestion and obstruction, sore throat, dysphonia, or respiratory distress, which can manifest as a life-threatening condition called acute respiratory distress syndrome (ARDS).

One important cause of ARI is infection with influenza viruses, which collectively cause a disease known as flu. Worldwide, flu causes 3-5 million cases of severe illness and 291,000-646,000 deaths annually, according to the World Health Organization and the U.S. Centers for Disease Control and Prevention. These totals can fluctuate widely depending on the particular strain of the influenza virus and other factors, and between 1976 and 2005 the annual deaths from flu in the U.S. ranged from 3,000 up to 49,000. The numbers of deaths per year between 2000 and 2006 were higher and ranged in the U.S. from 12,000 to 56,000. More recently, flu killed about 80,000 people in the U.S.

ARIs can be caused by multiple different pathogens other than influenza, including, as non-limiting examples, multiple strains of influenza (including avian influenza), respiratory syncytia virus (RSV) infection, coronaviruses (including Covid-19, severe acute respiratory syndrome (SARS), middle east respiratory syndrome (MERS)), retroviruses, parainfluenza virus infection, adenovirus infection, and metapneumovirus. Of these, RSV is the leading cause of respiratory infection worldwide. Making this problem even more challenging is the fact that these viruses have different strains that vary in their severity. For example, there are more than 50 distinct adenoviral serotypes able to cause a wide range of illnesses, from mild respiratory infections in young children to life-threatening multi-organ disease in people with a weakened immune system.

SARS-COV-2 has emerged as a global pandemic with an ever-increasing number of severe cases requiring invasive external ventilation that threatens to overwhelm health care systems (World Health Organization. Coronavirus disease (COVID-2019) situation reports. See who.int/emergencies/disease/novel-coronavirus-2019/situation-reports). While it remains unclear why COVID-19 patients experience a spectrum of clinical outcomes ranging from asymptomatic to severe disease, the salient features of COVID-19 pathogenesis and mortality are rampant inflammation and CRS leading to ARDS (Mehta, P. et al. COVID-19: consider cytokine storm syndromes and immunosuppression. Lancet 395, 1033-1034 (2020); Qin, C. et al. Dysregulation of immune response in patients with COVID-19 in Wuhan, China. Clin. Infect. Dis. (2020)). Indeed, excessive immune cell infiltration into the lung, cytokine storm, and ARDS have previously been described as defining features of severe disease in humans infected with the closely related betacoronaviruses SARS-COV and MERS-COV (Channappanavar, R. & Perlman, S. Pathogenic human coronavirus infections: causes and consequences of cytokine storm and immunopathology. Semin Immunopathol 39, 529-539 (2017); Nicholls, J. M. et al. Lung pathology of fatal severe acute respiratory syndrome. Lancet 361, 1773-1778 (2003)).

Viruses that infect the body outside the respiratory system can also be deadly, such as Marburg virus, Ebola viral strains, Hantaviruses, Bird flu virus, Lassa virus, Junin virus, Crimea-Congo fever virus, Macjupo virus, Kyasanur Forest, Dengue fever, Rabbit fever (tularemia), Rotavirus, Cholera, Plague, Toxic Shock syndrome, Meningococcal meningitis, Staphylococcus aureus (including MRSA), Vibrio illness, Hendra virus, and Nipah virus, as non-limiting examples.

Lethal infections may also be of bacterial origin. Non-limiting examples of such lethal bacterial infections include Shigella toxin-producing E. coli, Yellow fever, Rocky Mountain spotted fever, Diphtheria, pneumococcal pneumonia, and Legionnaire's disease, Streptococcus spp., Staphylococcus spp., Salmonella spp., Pseudomonas spp., Clostridium spp., Vibrio spp., Mycobacterium spp., or Haemophilus spp. Additional examples of bacteria that can cause a disease involving hypercytokinemia are known in the art. The diseases that can result from bacterial infections and that may manifest with damage to the body caused by hyper activation of the immune system, and can be followed by immunosuppression, include but are not limited to bacteremia, bacterial sepsis, pneumonia, cellulitis, meningitis, erysipelas, infective endocarditis, necrotizing fasciitis, prostatitis, pseudomembranous colitis, pyelonephritis, or septic arthritis.

With this diversity of pathogens able to cause mortality and significant morbidity, and the diversity of disease manifestations, it is not surprising that general treatments for this problem have been difficult to discover. It is also the case that treatments for individual infections have been difficult to discover. In the case of virus infections, there are relatively few viruses for which pharmacological treatments exist. The most effective treatments for viruses are vaccinations, which must be in place before infection occurs in order to be effective. In the case of bacterial infections, the question is whether there is an antibiotic available that is able to defeat that particular bacterial infection. Frequently, there is no antibiotic that is particularly effective. In addition, the medical community is generally lacking in treatments for physiological insults that cause immune activation excessive enough to potentially be lethal.

Thus, there is a need in the art for compositions and methods for treating a subject infected with a pathogen that is capable of causing death within the relatively short time period (e.g., less than six weeks) or that is capable of causing death by initiating treatment when a decline in health has already begun. There is also a need for compositions and methods for treating a subject infected with a pathogen that causes serious co-morbidities (e.g., a disruption of any of a variety of normal tissue functions). There is also a need in the art for compositions and methods for treating of other physiological insults (e.g., graft-versus-host disease, sepsis, radiation exposure, etc.) that can cause immune hyper-activation and/or immune exhaustion, such as those that occur with severe or critical pathogen infections. The present invention satisfies these unmet needs.

### SUMMARY OF THE INVENTION

In one aspect, the present invention is directed, in part, to a method of preventing death and severe morbidity induced by a pathogen. In another aspect, the present invention provides a method of preventing, alleviating, or treating an excessive immune activation (e.g., hypercytokinemia, also known as a cytokine storm and as cytokine release syndrome) induced by a pathogen or another inducer of immune exhaustion and/or excessive immune activation.

In one aspect, the present invention provides a method of preventing death of a subject caused by an exposure to an infectious pathogen. In some embodiments, the infectious pathogen is capable of causing a severe or critical disruption or dysfunction in a normal tissue function. In some embodiments, the infectious pathogen is capable of causing death within about 4 weeks after infecting the subject.

In one embodiment, the administration of the therapeutically effective amount of the pharmaceutical composition is transient and continued for 6 weeks or less.

In one embodiment, the administration of the therapeutically effective amount of the pharmaceutical composition is initiated before the infectious pathogen causes the severe or critical disruption or dysfunction in the normal tissue function in the subject.

In one aspect, the present invention provides a method of preventing, alleviating, or treating a tissue damage, tissue dysfunction, or any combination thereof in a subject in need thereof. In some embodiments, the tissue damage, tissue dysfunction, or any combination thereof is caused by an exposure to an infectious pathogen capable of causing a severe or critical disruption in a normal tissue function.

In some embodiments, the method reduces or reverses the tissue damage, tissue dysfunction, or any combination thereof in the subject.

In one aspect, the present invention provides a method of preventing, alleviating, or treating a mitochondrial damage, mitochondrial dysfunction, lysosomal damage, lysosomal dysfunction, or any combination thereof in a subject in need thereof.

In some embodiments, the mitochondrial damage, mitochondrial dysfunction, lysosomal damage, lysosomal dysfunction, or any combination thereof is caused by an exposure to an infectious pathogen capable of causing a severe or critical disruption in a normal tissue function. In other embodiments, the mitochondrial damage, mitochondrial dysfunction, lysosomal damage, lysosomal dysfunction, or any combination thereof is caused by an exposure to an insult capable of compromising an immune system function (e.g., a pathological insult that causes compromised immune system function, such as hyper-activation of the immune system or immune exhaustion), an infectious pathogen capable of causing a severe or critical disruption in a normal tissue function, or any combination thereof.

In one aspect, the present invention provides a method of preventing, alleviating, or treating an axonal damage or dysfunction in a subject in need thereof.

In some embodiments, the axonal damage or dysfunction is caused by an exposure to an infectious pathogen capable of causing a severe or critical disruption in a normal tissue function. In other embodiments, the axonal damage or axonal dysfunction is caused by an exposure to an insult capable of compromising an immune system function (e.g., a pathological insult that causes compromised immune system function, such as hyper-activation of the immune system or immune exhaustion), infectious pathogen capable of causing a severe or critical disruption in a normal tissue function, or any combination thereof.

In one aspect, the present invention provides a method of preventing, alleviating, or treating at least one gait abnormality in a subject in need thereof.

In some embodiments, the at least one gait abnormality is caused by an exposure to an infectious pathogen capable of causing a severe or critical disruption or dysfunction in a normal tissue function. In other embodiments, the at least one gait abnormality is caused by an exposure to an insult capable of compromising an immune system function (e.g., a pathological insult that causes compromised immune system function, such as hyper-activation of the immune system or immune exhaustion), infectious pathogen capable of causing a severe or critical disruption in a normal tissue function, or any combination thereof.

In one aspect, the present invention provides a method of preventing death in a subject in need thereof. In some embodiments, the death is preceded by a hyper-activation of the immune system, hypercytokinemia, cytokine storm, cytokine release syndrome, immune system exhaustion, or any combination thereof caused, for example, by a graft-versus-host disease, radiation, sepsis, or an exposure to an infectious pathogen capable of causing a severe or critical disruption in a normal tissue function. In other embodiments, the death is preceded by a hyper-activation of the immune system, hypercytokinemia, cytokine storm, cytokine release syndrome, immune system exhaustion, or any combination thereof.

In one aspect, the present invention provides a method of preventing, reducing, or reversing a tissue damage, tissue dysfunction, or any combination thereof in a subject in need thereof. In some embodiments, the tissue damage, tissue dysfunction, or any combination thereof is caused by a hyper-activation of the immune system, hypercytokinemia, cytokine storm, cytokine release syndrome, immune system exhaustion, or any combination thereof caused by a graft-versus-host disease, sepsis, exposure to radiation (e.g., ionizing radiation, lethal radiation, etc.), exposure to an infectious pathogen, or any combination thereof.

In one aspect, the present invention provides a method of restoring, improving, or enhancing at least a portion of a tissue function in a subject in need thereof. In some embodiments, the tissue function was reduced by an exposure to an infectious pathogen capable of causing a severe or critical disruption in a normal tissue function, graft-versus-host disease, sepsis, radiation, or any combination thereof.

In one aspect, the present invention provides a method of preventing, alleviating, or treating a hyper-activation of the immune system, hypercytokinemia, cytokine storm, cytokine release syndrome, immune system exhaustion, or any combination thereof in a subject in need thereof. In some embodiments, the hyper-activation of the immune system, hypercytokinemia, cytokine storm, cytokine release syndrome, immune system exhaustion, or any combination thereof is caused by a graft-versus-host disease, sepsis, radiation, or an exposure to an infectious pathogen capable of causing a severe or critical disruption in a normal tissue function.

In one aspect, the present invention provides a method of preventing, alleviating, or treating the morbidity or mortality caused by a graft-versus-host disease (e.g., the immune dysfunction occurring in a graft-versus-host disease) in a subject in need thereof.

In one aspect, the present invention provides a method of preventing, alleviating, or treating the morbidity or mortality caused by a sepsis and/or sepsis in a subject in need thereof. In one embodiment, the sepsis is caused by exposure to an infectious pathogen. In one embodiment, the sepsis is a result of pathogen infections that are not controlled because the immune system has been overwhelmed.

In various embodiments, the method comprises administering to the subject a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof.

In some embodiments, the pharmaceutical composition comprises at least one potassium channel blocker comprising 4-aminopyridine, a derivative of 4-aminopyridine, or a combination thereof.

In some embodiments, the derivative of 4-aminopyridine is a compound having the structure of Formula (I)

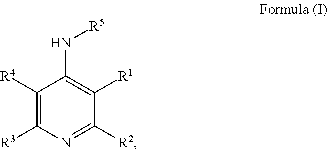

or an analog, a racemate, a tautomer, an isomer, an enantiomer, a diastereomer, a prodrug, or a pharmaceutically acceptable salt thereof.

In some embodiments, R¹, R², R³, R⁴, and R⁵ are each independently selected from hydrogen, halogen, C₁-C₆ alkyl (e.g., methyl, ethyl, propyl, butyl, pentyl, hexyl), amine, hydroxyl, alkoxy (e.g., methoxy, ethoxy, propoxy, butoxy, pentoxy, hexoxy), carboxyl, or any combination thereof. In one embodiment, C₁-C₆ alkyl is a C₁-C₆ haloalkyl (e.g., trifluoromethyl). For example, in some embodiments, R¹, R², R³, R⁴, and R⁵ are each independently selected from hydrogen, halogen, amine, hydroxyl, carboxyl, methyl, methoxy, trifluoromethyl, or any combination thereof.

In some embodiments, R¹, R², R³, R⁴, and R⁵ are optionally substituted.

In some embodiments, the therapeutically effective amount of the pharmaceutical composition is administered to the subject at the time when exposure to an infectious pathogen capable of causing severe or critical disruptions in normal tissue function, exposure to radiation (e.g., ionizing radiation, lethal radiation, etc.), or cell transplantation that could cause graft-versus-host disease, first occurs.

In some embodiments, the therapeutically effective amount of the pharmaceutical composition is administered to the subject after the time when exposure to an infectious pathogen capable of causing severe or critical disruptions in normal tissue function, exposure to radiation (e.g., ionizing radiation, lethal radiation, etc.), or cell transplantation that could cause graft-versus-host disease, first occurs.

In some embodiments, the therapeutically effective amount of the pharmaceutical composition is administered to the subject after the time when exposure to an infectious pathogen capable of causing severe or critical disruptions in normal tissue function, exposure to. radiation (e.g., ionizing radiation, lethal radiation, etc.), or cell transplantation that could cause graft-versus-host disease, first occurs but before the time when severe or critical changes in normal tissue function are apparent.

In some embodiments, the therapeutically effective amount of the pharmaceutical composition is administered to the subject after the time when exposure to an infectious pathogen capable of causing severe or critical disruptions in normal tissue function, exposure to radiation (e.g., ionizing radiation, lethal radiation, etc.), or cell transplantation that could cause graft-versus-host disease, first occurs but at or after the time when severe or critical changes in normal tissue function are apparent.

In some embodiments, the therapeutically effective amount of the pharmaceutical composition is repeatedly administered to the subject for between about 1 day to about 100 years.

In some embodiments, the therapeutically effective amount of the pharmaceutical composition is administered to the subject systematically, locally, or a combination thereof.

In some embodiments, the therapeutically effective amount of the pharmaceutical composition is administered to the subject by an intraperitoneal injection, intravenous injection, intramuscular injection, intrathecal injection, subcutaneous injection, sublingual administration, inhalation, oral administration, transdermal administration, administration to an outer portion of the body in the form of a liquid, administration to an outer portion of the body in the form of a salve, administration to an outer portion of the body in the form of a bandage, or any combination thereof.

In some embodiments, the therapeutically effective amount of the pharmaceutical composition is administered to the subject at a dose of between about 1 mg/day to about 1,000 mg/day of the potassium channel blocker. In some embodiments, the therapeutically effective amount of the pharmaceutical composition is administered to the subject at a dose of between about 2.5 mg/day to about 40 mg/day of the potassium channel blocker. In some embodiments, the therapeutically effective amount of the pharmaceutical composition is administered to the subject at a dose of between about 40 mg/day to about 100 mg/day of the potassium channel blocker.

In some embodiments, the therapeutically effective amount of the pharmaceutical composition is co-administered with at least one anticonvulsant agent or a composition thereof. In some embodiments, the at least one anticonvulsant agent is barbiturate, benzodiazepine, bromide, carbamate, carboxamide, fatty acid, fructose or a derivative thereof, γ-aminobutyric acid (GABA) or an analog thereof, hydantoin, oxazolidinedione, proprionate, pyrimidinedione, pyrrolidine, succinimide, sulfonamide, triazine, urea, valproylamide, or any combination thereof.

In some embodiments, the method further enhances cell survival, reduces scarring, or any combinations thereof.

In some embodiments, the method further enhances a repair or regeneration of endogenous stem cells, enhances a repair or regeneration of transplanted stem cells, enhances a repair or regeneration of progenitor cells, promotes a neural cell generation, enhances cell survival, reduces scarring, decreases lesion size, decreases oxidative damage, or any combinations thereof.

In one aspect, the present invention also provides a method of identifying a subject responsive to administration of 4-aminopyridine or a derivative thereof to prevent, alleviate, or treat a tissue damage or tissue dysfunction caused by an exposure to an infectious pathogen, graft-versus-host disease, sepsis, radiation, or any combination thereof. In some embodiments, the method comprises the steps of: a) administering to the subject between 1 to 5 therapeutically effective amounts of a pharmaceutical composition comprising a 4-aminopyridine, a derivative of 4-aminopyridine, or a combination thereof; b) evaluating the symptoms of the tissue damage or tissue dysfunction caused by exposure to an infectious pathogen capable of causing severe or critical disruptions in normal tissue function, or by graft-versus-host disease in a subject in need thereof; and c) identifying the subject as responsive to 4-aminopyridine administration to prevent, alleviate, or treat the tissue damage or tissue dysfunction caused by the exposure to the infectious pathogen, graft-versus-host disease, or any combination thereof when the symptoms of the tissue damage or tissue dysfunction caused by the exposure to the infectious pathogen, graft-versus-host disease, or any combination thereof in the subject improved.

In some embodiments, the infectious pathogen is a viral pathogen, bacterial pathogen, fungal pathogen, or any combination thereof.

In one embodiment, the infectious pathogen is a viral pathogen. In some embodiments, the viral pathogen is a respiratory virus, non-respiratory virus, or a combination thereof. In one embodiment, the viral pathogen is a respiratory virus.

In one embodiment, the infectious pathogen is a bacterial pathogen. In some embodiments, the infectious pathogen is a bacterial respiratory pathogen, bacterial non-respiratory pathogen, or a combination thereof. In some embodiments, the infectious pathogen is bacterial and a respiratory pathogen.

In one embodiment, the infectious pathogen is a fungal pathogen.

In some embodiments, the infectious pathogen infectious pathogen is an influenza virus, avian influenza virus, respiratory syncytia virus, coronavirus, Covid-19 virus, severe acute respiratory syndrome (SARS) virus, middle east respiratory syndrome (MERS) virus, retrovirus, parainfluenza virus, adenovirus, metapneumovirus, marburg virus, Ebola virus, hantaviruse, lassa virus, junin virus, Crimea-Congo fever virus, macjupo virus, kyasanur forest virus, dengue fever virus, rabbit fever virus, tularemia virus, rotavirus, cholera pathogen, plague pathogen, pathogen causing toxic shock syndrome, pathogen causing meningococcal meningitis, Staphylococcus aureus (including MRSA), pathogen causing vibrio illness, Hendra virus, Nipah virus, Shigella toxin-producing E. coli, yellow fever pathogen, rocky mountain spotted fever pathogen, diphtheria pathogen, pneumococcal pneumonia pathogen, legionnaire's disease pathogen, Streptococcus spp., Staphylococcus spp., Salmonella spp., Pseudomonas spp., Clostridium spp., Vibrio spp., Mycobacterium spp, or Haemophilus spp., or any combination thereof.

In some embodiments, the tissue damage is a multi-tissue damage, multi-organ tissue damage, or any combination thereof.

In some embodiments, the tissue damage is a damage of a motor function, sensory function, cognitive function, visual function, auditory function, kidney function, respiratory function, hematopoietic system function, normal skin function, salivary gland function, liver function, gall bladder function, gastrointestinal (GI) function, sexual function, or any combination thereof.

In some embodiments, the tissue damage is a kidney tissue damage, liver tissue damage, heart tissue damage, lung tissue damage, brain tissue damage, central nervous system damage, peripheral nerve tissue damage, peripheral neuropathy, nephropathy, neutropenia, gastrointestinal tract tissue damage, gut tissue damage, visual system tissue damage, auditory system tissue damage, skin tissue damage, bladder tissue damage, reproductive system tissue damage, hematopoietic system tissue damage, immune system tissue damage, or any combination thereof.

In some embodiments, the subject has a compromised immune function due to a graft-versus-host disease, sepsis, exposure to radiation (e.g., ionizing radiation, lethal radiation, etc.), infection caused by an infectious pathogen, or any combination thereof. In some embodiments, the infectious pathogen is capable of causing a severe or critical disruption in a normal tissue function, wherein a normal tissue function required for survival is reduced by an exposure to the infectious pathogen. For example, in one embodiment, the subject has a graft-versus-host disease.

In some embodiments, the subject developed a hyper-activation of the immune system, hypercytokinemia, cytokine storm, cytokine release syndrome, immune system exhaustion, or any combination thereof caused by the exposure to the infectious pathogen.

In some embodiments, the subject has at least one mutation compromising a function of the subject's immune system.

In some embodiments, the immune system is weakened by genetic mutation, exposure to ionizing radiation, chemotherapy, aging, Alzheimer's disease, sepsis, or other factors that interfere with the normal ability of the immune system to prevent infection-induced morbidity or mortality.

In some embodiments, the subject has ataxia. In some embodiments, the ataxia is a heritable ataxia, such as ataxia telangiectasia or another heritable ataxia.

### BRIEF DESCRIPTION OF THE DRAWINGS

The following detailed description of various embodiments of the present invention will be better understood when read in conjunction with the appended drawings. For the purpose of illustrating the present invention, there are shown in the drawings illustrative embodiments. It should be understood, however, that the present invention is not limited to the precise arrangements and instrumentalities of the embodiments shown in the drawings.

FIG. 1, comprising FIG. 1A and FIG. 1B, depicts representative results demonstrating that ataxia telangiectasia mutated (ATM) knockout (KO) mice were more susceptible to lethal influenza infections. FIG. 1A depicts representative results demonstrating that ATM KO mice (solid line) were more vulnerable to lethal effects of influenza infection than wildtype (WT) mice (dashed line). FIG. 1B depicts representative results demonstrating ATM KO mice (filled triangles line) showed greater weight loss post-infection than WT mice (filled circles), a co-morbidity of infection. Uninfected mice followed for the same time showed no weight loss (un-filled circles and triangles).

FIG. 2, comprising FIG. 2A and FIG. 2B, depicts representative results demonstrating that 4-aminopyridine (4-AP) treatment prevented influenza-induced lethality in ATM KO animals. FIG. 2A depicts representative results demonstrating that ATM KO mice infected with influenza virus showed modestly improved survival with corticosteroid treatment (dashed line) over untreated virus-infected ATM KO mice in FIG. 1A. In contrast, 4-AP treated mice showed complete rescue from death (solid line). FIG. 1B depicts representative results demonstrating that weight loss in infected mice was also attenuated with 4-AP treatment (solid line), but not by treatment with corticosteroid (dashed line).

FIG. 3, comprising FIG. 3A and FIG. 3B, depicts representative results demonstrating that ATM KO mice exposed nasally to a nonlethal, non-neurotropic, influenza virus infection developed a post-infection impairment in motor function, as detected by rotarod analysis, which is used to detect defects in motor strength in the ability to hold on to the rod and defects in gait related to the ability to increase walking speed as the speed of the rod movement increases. WT and Atm null (AT [M]) animals were exposed (nasally) to a non-neurotropic, non-lethal influenza virus and were randomly assigned to either treatment with 4-AP at 1.5 mg/kg for 5 days (3 days post-infection to 7 days post-infection). FIG. 3A depicts a representative experimental regime, in which mice were exposed to infection at Day 0. At Day 3, treatment of mice with 4-AP (1.5 mg/kg) or saline was initiated and was continued for 5 days (i.e., until Day 7 post-infection). Mice rapidly became very ill, and showed substantial weight loss by 3 days after infection. This viral preparation did not cause lethality in ATM KO animals and loss of weight was comparable to the WT cohort. To determine whether 4-AP prevents development of motor defects in virus-exposed ATM KO mice, a rotarod analysis was conducted. Animals were exposed to an accelerating rod, three consecutive runs were conducted and the time to fall was determined. FIG. 3B depicts representative results of motor function analysis where the speed at which mice fall from the rotarod was determined, and was found to be significantly decreased in virus-exposed mice treated with saline but not in virus-exposed mice treated with 4-AP. Two way Anova p*=0.02.

### DETAILED DESCRIPTION

There are many challenges in developing means of preventing damage caused by pathogen infections that have severe or critical consequences. Such infections cause activation of the immune system, which utilizes cell-based, protein-based and small-molecule based responses to suppress the pathogen. In addition, infected cells produce their own countermeasures, ranging from processes occurring within the cell and secreted substances that will decrease infection of other cells and/or activate the host defenses in order to limit the activities of the pathogen. Paradoxically, the reactions of the immune system may exacerbate the damage caused by infectious pathogens in two ways. If the immune system is hyper-activated in any of several different ways, this can itself be a cause of tissue damage. If the function of the immune system is compromised so that the immune response is inadequate to control the pathogen, then this can exacerbate damage caused by the infectious pathogen itself.

Examples of multiple of these potential disruptions are provided in the illustrative examples that follow. At the general level, disruptions in normal cellular function that have been suggested to be relevant to the pathogen-induced morbidity and mortality can include dysfunction of intracellular organelles (e.g., mitochondria, lysosomes, microtubules), production of reactive oxidative species and other potentially toxic metabolites, alterations in multiple aspects of cellular metabolism, the production of defense reactions in infected cells, the activation of surrounding cells (e.g., due to the presence of cell death in the environment or due to the production of signals that indicate the presence of a potential threat), the activation of multiple aspects of the immune response, the production of immune cytokines, the attack by components of the immune system on the pathogen and on infected cells, the production by the pathogen of its own defense systems, the hijacking of normal cellular function by the pathogen and multiple other processes. All of these processes may lead to dysfunction of the infected tissue(s), systemic spread of the virus, and widespread tissue and organ damage as a result of pathogen infection and also as a potential result of the attempts of the body to fight the pathogen. The attempts to fight the pathogen can create a positive feedback loop of damage, in which the spread of damage further activates more damage response, which in turn further increases cellular, organ and tissue dysfunction. Even when the damage is limited to a single tissue, there are tissues (such as the lungs, for example) in which disruption of normal function is by itself sufficient to cause death or significant morbidity.

These damaging endpoints can also be reached by other means, such as exposure to radiation (e.g., ionizing radiation, lethal radiation, etc.) or development of graft-versus-host disease or sepsis (as non-limiting examples). Although the specific inducer of injurious immune response may be different, the complexity of the immune response and the need for improved treatments are very similar.

The existence of so many possible dysfunctions caused by infectious pathogens able to cause severe or critical outcomes indicates the challenges of finding treatments for these problems. It is not clear whether there are particular changes that are so critical that reversing them individually would rescue from mortality or morbidity. This has in fact been the case for many attempts to prevent severe or critical outcomes following pathogen infection, with death and morbidity continuing to occur frequently despite targeting of specific changes occurring as a consequence of these infections. Although some compounds have shown initial promise, none are able to meet the medical needs that exist in these situations.

The novelty of using 4-AP in the treatment of the consequences of exposure to a pathogen (e.g., an infectious pathogen) or another inducer of immune exhaustion and/or excessive immune activation capable of causing severe or critical disruptions in normal tissue function, lies at least in part in the cause of the damage and the unexpected ability of 4-AP to be useful in treating such damage. These threats can rapidly involve multiple organ systems and cell types, can be extremely variable in their consequences, and in severe-to-critical situations can induce damage much greater and more widespread than that seen in previous examination of the utility of 4-AP as a potential therapeutic agent.

Thus, the present invention is based, in part, on the unexpected results that treatment with 4-AP effectively prevented influenza virus-induced death, as compared with the lethality seen in animals that were infected with an identical dose of virus and were treated with an equal volume of saline. Moreover, treatment with 4-AP was more effective in preventing influenza-virus induced death than treatment with corticosteroids.

The present invention is also based, in part, on the unexpected results that treatment with 4-AP also ameliorated influenza virus-induced weight loss as compared with a control (i.e., animals that were infected with an identical dose of virus and were treated with an equal volume of saline). Moreover, treatment with 4-AP was more effective in preventing influenza-virus induced death than treatment with corticosteroids.

The present invention is also based, in part, on the unexpected results that treatment with 4-AP effectively prevented influenza virus-induced declines in motor function, as compared with the declines seen in animals that were infected with an identical dose of virus and were treated with an equal volume of saline. Such declines in motor function following a febrile and/or respiratory infection are frequently seen in children with AT, and occur in ATM KO mice following infection with influenza virus and after respiratory exposure to lipopolysaccharide (as a model of respiratory bacterial infections).

The present invention is also based, in part, on the unexpected results that the benefits of 4-AP treatment occurred despite the fact that treatments were carried out in mice with a genetic mutation known to render them more susceptible to respiratory infection and with several immune defects. Although mice with knockout mutations in the ATM gene are not immune-incompetent to the extent of mice lacking entire components of the immune system (as in, e.g., Nod-scid gamma mice), they nonetheless have serious defects. Mice with knockout mutations in the ATM gene have an immune defect that is a partial defect, more akin to defects caused by, as non-limiting examples, other genetic causes, exposure to ionizing radiation, exposure to chemotherapeutic agents or other toxic insults, stress, aging, Alzheimer's disease, and/or concomitant infections (e.g., with HIV as a non-limiting example). Thus, rescue of these ATM KO mice provides a particularly potent example of the ability of 4-AP treatment to prevent adverse effects of pathogen infection even in individuals with compromised immune function. Such efficacy suggests that benefits would be even greater in individuals in whom the immune system is not compromised.

As transient 4-AP exposure causes stopping the process of decline, such that treated individuals did not resume their decline when treatment was stopped after several days, the present invention also relates, in part, to compositions and methods for treating a subject infected with a pathogen that is capable of causing death within a relatively short time period such that the treatment is able to stop the process of deterioration so effectively that it does not continue on its prior course after the treatment is stopped.

In one embodiment, the compositions and methods of treatment may provide any of a number of different benefits, including but not limited to decreasing viral load, preventing further increases in viral load, preventing, decreasing, or reversing excess immune activation, enhancing normal immune function, preventing tissue damage caused by excess immune action, and/or restoring normal physiological function in the infected individual.

In some embodiments, the compositions and methods of treatment may prevent or reverse an immune system exhaustion (e.g., sepsis).

In other embodiments, the methods may be used to treat severe morbidity and mortality in situations of radiation exposure, and particularly radiation with an additional injury (i.e., radiation-combined injury), in which affected individuals show hypercytokinemia, changes in immune function like those seen in other insults pertinent to the present invention, and increased vulnerability to pathogen infection and sepsis.

The method can include administering to the subject a pharmaceutical composition comprising a potassium channel blocker, which is preferably 4-AP, a derivative thereof, or a combination thereof. In some embodiments, the damage caused by the pathogen infection can be multi-site and/or multi-organ. In some embodiments, the method can include administering to the subject a therapeutically effective amount of a pharmaceutical composition comprising 4-AP, a derivative thereof, or a combination thereof. In some embodiments, the pharmaceutical composition can be formulated to provide sustained release of the 4-AP, a derivative of 4-AP, or a combination thereof. In certain embodiments, 4-AP or a derivative thereof can be represented by a structure according to Formula (I)

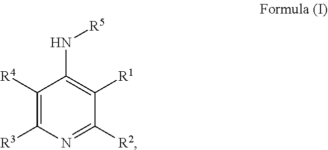

or an analog, a racemate, a tautomer, an isomer, an enantiomer, a diastereomer, a prodrug, a pharmaceutically acceptable salt, or a derivative thereof. In some embodiments, the derivative of 4-AP comprises 3,4-diaminopyridine, 3-hydroxy-4-AP, or a combination thereof.

In some embodiments, the pharmaceutical composition can be administered to the subject by injection, intraperitoneal injection, intravenous injection, intramuscular injection, intrathecal injection, subcutaneous injection, sublingual administration, inhalation, oral administration, transdermal administration, implantation, insertion of a device into the subject, or any combination thereof.

In some embodiments, the pharmaceutical compositions can be administered in combination with an additional therapeutic agent in order to provide protection against the ability of at least one potassium channel blocker to cause convulsions in rare individuals if the serum levels exceed defined thresholds. For example, the pharmaceutical composition can be administered with an anticonvulsant. The anticonvulsant can be selected from lamotrigine, gabapentin, valproic acid, topiramate, famotodine, phenobarbital, diphenylhydantoin, phenytoin, mephenytoin, ethotoin, mephobarbital, primidone, carbamazepine, ethosuximide, methsuximide, phensuximide, trimethadione, benzodiazepine, phenacemide, acetazolamide, progabide, clonazepam, divalproex sodium, magnesium sulfate injection, metharbital, paramethadione, phenytoin sodium, valproate sodium, clobazam, sulthiame, dilantin, diphenylan, and L-5-hydroxytrytophan, or any combination thereof.

The pharmaceutical compositions may be administered at various times, depending on the goal of treatment. In order to prevent the severe or critical consequences of the pathogen infection and/or of the hyper-activation of the immune system, the treatment may be administered prophylactically, beginning after infection and after symptoms of the infection are apparent but before severe or critical decline occurs. In order to slow or reverse the progress of the damage, or to overcome the symptoms of the damage, treatment may be administered at any time when damage has begun to become apparent, as determined, for example, by the development of clinically relevant symptoms.

The pharmaceutical composition can be administered repeatedly throughout the duration of the infection if the goal is to prevent manifestation of injury. In some cases, and particularly if the treatment is being used to provide symptomatic relief, then treatment can be continued for as long as symptoms persist.

The subject in need of treatment can be administered a dose of from about 5 mg/day to about 100 mg/day of 4-AP, 4-AP derivative, or a combination thereof. In certain embodiments, the subject can be administered a dose of from about 5 mg/day to about 40 mg/day or about 40 mg/day to about 100 mg/day of 4-AP, 4-AP derivative, or a combination thereof. In some embodiments, the dosages of 4-AP or the derivative thereof may be increased or decreased from these levels depending on the effective therapeutic dosage range from each specific agent.

It will be also readily apparent to one skilled in the art that the quantity and frequency of the administration of the potassium channel blocker or the pharmaceutical composition thereof will depend on many factors including, but not limited to, the identity of the potassium channel blocker, type and severity of the subject's disease or disorder, condition of the subject, age of the subject, gender of the subject, overall health of the subject, weight of the subject, and other factors, although appropriate dosages may be determined by clinical trials. Thus, the invention should not be construed to be limited to any particular dosage regime and the precise dosage and composition to be administered to any subject will be determined by the attending physician taking all other factors about the subject into account.

In some embodiments, the methods described herein can be used for treating damage caused by a pathogen (e.g., pathogen infection) or another inducer of immune exhaustion and/or excessive immune activation in a subject. For example, the methods described herein can be used for treating the effects of exposure to viral pathogens, bacterial pathogens, and/or fungal pathogens. The individual treated may be healthy at the time of pathogen infection, or may have a recognized contributor to vulnerability, such as a mutation, metabolic condition, concurrent infection or other illness, chronic stress, aging, Alzheimer's disease, exposure to ionizing radiation, exposure to chemotherapeutic agents or other toxic insults, concomitant infections (e.g., with HIV as a non-limiting example), immunosuppression and/or treatment with medicaments that have the side effect of increasing vulnerability to pathogen infection.

The inducers of immune exhaustion and/or excessive immune activation may also be individual components of the immune cascade or immune regulation that cause hyper-activation of different components of the immune system as manifested, for example, by changes in numbers and or activation of specific cell populations, expression of hypercytokinemia, cytokine release syndrome, immune system exhaustion (e.g., sepsis), macrophage activation syndrome, graft-versus-host disease or other changes associated with hyper-activation of the immune system or biological or chemical warfare agents that have the consequence of causing hyper-activation of the immune system.

In some embodiments, the damage can be most notable in one system of the body, such as in acute respiratory infections. In other embodiments, the damage may be manifested in more than one tissue, including, for example, the pulmonary system, the peripheral nervous system, the central nervous system, the visual system, the auditory system, the hematopoietic system, the gastrointestinal system, the bladder, the heart, skeletal muscle, hair follicles, skin, vasculature, the salivary gland or a combination of any of these tissues.

In some embodiments, the methods described herein can restore at least a portion of lost motor function or/and sensory function in the subject, enhance repair and regeneration of cells such as by promoting stem or precursor cell generation, enhance cell survival, reduce scarring, or combinations thereof, as compared to an untreated subject. In additional embodiments, the methods described herein can restore at least a portion of function of the damaged tissue, enhance repair and regeneration, enhance cell survival, reduce scarring, decrease other aspects of tissue damage or combinations thereof, as compared to an untreated subject. In further embodiments, the methods described herein can restore at least a portion of function of the damaged tissue, enhance repair and regeneration, enhance cell survival, reduce scarring, or combinations thereof, as compared to an untreated subject.

In some embodiments, the methods disclosed herein can be used for preventing or treating muscle atrophy that is associated with hyper-activation of the immune system.

In some embodiments, the methods disclosed herein can be used for preventing, ameliorating, reversing, or otherwise treating tissue dysfunction caused by infection with pathogens capable of causing death within several days to several weeks of infection, or tissue dysfunction caused by other insults capable of compromising an immune system function (e.g., pathological insults capable of causing immune system hyper-activation and/or immune system suppression). In some embodiments, the tissue dysfunction may occur in, for example, the hematopoietic system, hair follicles, cells in the mouth, digestive tract, reproductive system, and cells in the heart, kidneys, bladder, salivary glands, auditory system, visual system, lungs, nervous system, or any combination thereof.

In one aspect, the invention provides methods of preventing, alleviating, and/or treating a tissue damage caused by exposure to an infectious pathogen capable of causing severe or critical disruptions in normal tissue function, or to such other insults capable of compromising an immune system function (e.g., pathological insults capable of causing excess immune activation or immune exhaustion) as graft-versus-host disease, sepsis, and/or radiation exposure, and where the tissue damage includes, but is not limited to, tissue dysfunction, mitochondrial dysfunction, muscle atrophy, neuropathy, nephropathy, or any combination thereof.

In one aspect, the present invention provides methods of reducing or reversing a tissue damage caused by exposure to an infectious pathogen capable of causing severe or critical disruptions in normal tissue function, or to such other insults capable of compromising an immune system function (e.g., pathological insults capable of causing excess immune activation or immune exhaustion) including, but not limited to, as graft-versus-host disease, sepsis, and/or radiation exposure.

In some embodiments, the tissue damage is a multi-tissue damage, multi-organ tissue damage, or any combination thereof. In some embodiments, the tissue damage is a kidney tissue damage, liver tissue damage, heart tissue damage, lung tissue damage, brain tissue damage, central nervous system damage, peripheral nerve tissue damage, peripheral neuropathy, nephropathy, neutropenia, gastrointestinal tract tissue damage, gut tissue damage, visual system tissue damage, auditory system tissue damage, skin tissue damage, bladder tissue damage, reproductive system tissue damage, hematopoietic system tissue damage, immune system damage, or any combination thereof.

In some embodiments, the tissue dysfunction is a motor dysfunction, sensory dysfunction, cognitive dysfunction, visual dysfunction, auditory dysfunction, kidney dysfunction, hematopoietic system dysfunction, respiratory system dysfunction, normal skin function, salivary gland dysfunction, liver dysfunction, gall bladder dysfunction, gastrointestinal (GI) dysfunction, sexual dysfunction, or any combination thereof.

In one aspect, the present invention provides methods of restoring, improving, and/or enhancing at least a portion of tissue function effected by exposure to an infectious pathogen capable of causing severe or critical disruptions in normal tissue function, or to such other insults capable of compromising an immune system function (e.g., pathological insults capable of causing excess immune activation or immune exhaustion) as graft-versus-host disease, sepsis, radiation exposure and/or where the effected tissue function includes, but is not limited to, myelination, tissue regeneration, cell survival, stem or progenitor cell generation, repair or regeneration of endogenous stem or progenitor cells, repair or regeneration of transplanted stem cells, repair or regeneration of progenitor cells, or any combination thereof.

In some embodiments, in a test sample obtained from the subject the severity of a disease may be determined by analysis of tissue function, changes in patient physiology, and/or changes in metabolic parameters. The presence of immune system hyper-activation or immune system exhaustion may be determined, as non-limiting examples, by analysis of changes in the cellular compositions and/or activation state of cellular components of the hematopoietic system, including the immune system, by analysis of local immune system activation and/or by assessment of levels of cytokines involved in hypercytokinemia and/or of CCL5/RANTES using tests and techniques well known to those skilled in the relevant art.

In some embodiments, the methods disclosed herein can be used to identify individuals who will benefit from a treatment with potassium channel blockers (e.g., 4-AP, a derivative of 4-AP, or any combination thereof). For example, in some embodiments, the present invention relates to a method of identifying a subject responsive to 4-AP administration to prevent, alleviate, or treat a tissue damage or tissue dysfunction caused by infection with a pathogen capable of causing lethality or severe morbidity in a subject in need thereof, the method comprising the steps of: a) administering to the subject between 1 to 5 therapeutically effective amounts of a pharmaceutical composition comprising 4-AP, a derivative of 4-AP, or a combination thereof; b) evaluating the symptoms of the tissue damage or tissue dysfunction caused by the pathogen infection in the subject; and c) identifying the subject as responsive to 4-AP administration to prevent, alleviate, or treat the tissue damage or tissue dysfunction caused by a pathogen infection when the symptoms of the tissue damage or tissue dysfunction caused by the pathogen infection in the subject improved.

In some embodiments, individuals manifesting dysfunction in one or more tissues following exposure to a pathogen capable of causing lethality or severe morbidity within several days to several weeks after infection may be treated with the methods disclosed herein for between one and ten days to determine if treatment provides improvement in tissue function. Thus, in some embodiments, the methods disclosed herein can be used to provide personalized targeting of therapies.

In some embodiments, individuals exposed to an infectious pathogen or other pathological circumstance that causes compromised immune function (e.g., graft-versus-host disease, radiation exposure, such as an exposure to ionizing radiation or a lethal radiation exposure, sepsis, stress, such as a chronic stress, aging, Alzheimer's disease, concomitant infection, such as an infection with HIV, exposure to chemotherapeutic agents or other toxic insults, etc.), and in whom one or more changes in tissue function caused by exposure to pathogen or pathological circumstance are present, are treated with between 1 to 5 treatments with a potassium channel blocker (e.g., 4-AP, derivative of 4-AP, or a combination thereof) in order to prognostically identify individuals in whom treatment should be continued for longer times. In some embodiments, if individuals show improvements in tissue function, then the longer-term methods disclosed herein are applied. In some embodiments, an individual with damage caused by an infectious pathogen or other pathological circumstances (e.g., graft-versus-host disease, radiation exposure, such as an exposure to ionizing radiation or a lethal radiation exposure, sepsis, stress, such as a chronic stress, aging, Alzheimer's disease, concomitant infection, such as an infection with HIV, exposure to chemotherapeutic agents or other toxic insults, etc.) and with symptoms in the realms of gait, pain, or nerve conduction velocity, respiratory insufficiency, or defective function of other organs is treated with between 1 to 5 treatments of 4-AP or a derivative thereof, and the symptoms are measured between 1-8 hours after initiation of treatment (i.e., within two half-lives of 4-AP in the serum). For example, in some embodiments, an individual with symptoms in the realms of gait, pain, or nerve conduction velocity, respiratory insufficiency, or defective function of other organs is treated with between 1 to 5 treatments of 4-AP or a derivative thereof, and the symptoms are measured between 1-8 hours after initiation of treatment (i.e., within two half-lives of 4-AP in the serum).

In some embodiments, the individual may have existing ailments worsened by the infectious pathogen. For example, emergence of gait abnormalities in an individual with a variety of ataxias or heritable ataxias may be stimulated by pathogen infection, and treatment with 4-AP or a derivative thereof is used to treat said individual according to the methods of the present invention.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### Definitions

Unless defined otherwise, all technical and scientific terms used herein have the same meaning as commonly understood by one of ordinary skill in the art to which the present invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “compound,” as used herein, unless otherwise indicated, refers to any specific chemical compound disclosed herein. In one embodiment, the term also refers to stereoisomers and/or optical isomers (including racemic mixtures) or enantiomerically enriched mixtures of disclosed compounds.

As used herein, the term “analog” or “analogue” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative can also be a small molecule that differs in structure from the reference molecule, but retains the essential properties of the reference molecule. An analog or derivative may change its interaction with certain other molecules relative to the reference molecule. An analog or derivative molecule may also include a salt, an adduct, tautomer, isomer, or other variant of the reference molecule.

The term “derivative” refers to a small molecule that differs in structure from the reference molecule, but retains the essential properties of the reference molecule. A derivative may change its interaction with certain other molecules relative to the reference molecule. A derivative molecule may also include a salt, an adduct, tautomer, isomer, or other variant of the reference molecule.

The term “tautomers” are constitutional isomers of organic compounds that readily interconvert by a chemical process (tautomerization).

The term “isomers” or “stereoisomers” refer to compounds, which have identical chemical constitution, but differ with regard to the arrangement of the atoms or groups in space.

The term “prodrug” refers to compounds that differ in structure from the reference molecule, but is chemically modified by a particular cellular process to ultimately become modified to retain the essential properties of the reference molecule or become the reference molecule. As used herein, the term “prodrug form” and its derivatives is used to refer to a drug that has been chemically modified to add and/or remove one or more substituents in such a manner that, upon introduction of the prodrug form into a subject, such a modification may be reversed by naturally occurring processes, thus reproducing the drug. Examples of prodrugs include, but are not limited to, esters, optionally substituted esters, branched esters, optionally substituted branched esters, carbonates, optionally substituted carbonates, carbamates, optionally substituted carbamates, thioesters, optionally substituted thioesters, branched thioesters, optionally substituted branched thioesters, thiocarbonates, optionally substituted thiocarbonates, sulfenyl thiocarbonates, optionally substituted sulfenyl thiocarbonates, 2-hydroxypropanoate ester, optionally substitute 2-hydroxypropanoate ester, S-thiocarbonate, optionally substituted S-thiocarbonate, dithiocarbonates, optionally substituted dithiocarbonates, thiocarbamates, optionally substituted thiocarbamates, oxymethoxycarbonyl, optionally substituted oxymethoxycarbonyl, oxymethoxycarbonate, optionally substituted oxymethoxycarbonate, oxymethoxythiocarbonyl, optionally substituted oxymethoxythiocarbonyl, oxymethylcarbonyl, optionally substituted oxymethylcarbonyl, oxymethylthiocarbonyl, optionally substituted oxymethylthiocarbonyl, oxymethoxythiocarbonate, optionally substituted oxymethoxythiocarbonate, L-amino acid esters, D-amino acid esters, oxymethoxy amino ester, N-substituted L-amino acid esters, N,N-disubstituted L-amino acid esters, N-substituted D-amino acid esters, N,N-disubstituted D-amino acid esters, sulfenyl, optionally substituted sulfenyl, sulfmyl, sulfonyl, sulfite, sulfate, sulfonamide, imidate, optionally substituted imidate, hydrazonate, optionally substituted hydrazonate, oximyl, optionally substituted oximyl, imidinyl, optionally substituted imidinyl, imidyl, optionally substituted imidyl, aminal, optionally substituted aminal, hemiaminal, optionally substituted hemiaminal, acetal, optionally substituted acetal, hemiacetal, optionally substituted hemiacetal, carbonimidate, optionally substituted carbonimidate, thiocarbonimidate, optionally substituted thiocarbonimidate, carbonimidyl, optionally substituted carbonimidyl, carbamimidate, optionally substituted carbamimidate, carbamimidyl, optionally substituted carbamimidyl, thioacetal, optionally substituted thioacetal, S-acyl-2-thioethyl, optionally substituted S-acyl-2-thioethyl, (acyloxybenzyl) ether, (acyloxybenzyl) ester, PEG ester, PEG carbonate, bis-(acyloxybenzyl) esters, optionally substituted bis-(acyloxybenzyl) esters, (acyloxybenzyl) esters, optionally substituted (acyloxybenzyl) esters, or BAB-esters, acetate, formate and benzoate derivatives of alcohol functional groups in the compounds. Methods of structuring a compound as prodrugs can be found in the book of Testa and Mayer, Hydrolysis in Drug and Prodrug Metabolism, Wiley (2006). Typical prodrugs form the active metabolite by transformation of the prodrug by hydrolytic enzymes, the hydrolysis of amide, lactams, peptides, carboxylic acid esters, epoxides or the cleavage of esters of inorganic acids.

As used herein, the term “alkyl,” by itself or as part of another substituent means, unless otherwise stated, a straight or branched chain hydrocarbon having the number of carbon atoms designated (i.e. C₁₋₆ means one to six carbon atoms) and includes straight, branched chain, or cyclic substituent groups. Examples include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. The term “alkyl,” unless otherwise noted, is also meant to include those derivatives of alkyl defined in more detail below, such as “heteroalkyl”, “haloalkyl” and “homoalkyl”.

As used herein, the term “substituted alkyl” means alkyl, as defined above, substituted by one, two or three substituents selected from halogen, —OH, alkoxy, —NH₂, —N(CH₃)₂, —C(═O) OH, trifluoromethyl, —C≡N, —C(═O)O(C₁-C₄)alkyl, —C(═O) NH₂, —SO₂NH₂, —C(═NH) NH₂, or —NO₂, preferably containing one or two substituents selected from halogen, —OH, alkoxy, —NH₂, trifluoromethyl, —N(CH₃)₂, or —C(═O) OH, more preferably selected from halogen, alkoxy, or —OH. Examples of substituted alkyls include, but are not limited to, 2,2-difluoropropyl, 2-carboxycyclopentyl, and 3-chloropropyl.

As used herein, the term “alkylene” by itself or as part of another molecule means a divalent radical derived from an alkane, as exemplified by (—CH₂—)ₙ. By way of example only, such groups include, but are not limited to, groups having 24 or fewer carbon atoms such as the structures —CH₂CH₂— and —CH₂CH₂CH₂CH₂—. The term “alkylene,” unless otherwise noted, is also meant to include those groups described below as “heteroalkylene.”

As used herein, the terms “alkoxy,” “alkylamino” and “alkylthio” are used in their conventional sense, and refer to alkyl groups linked to molecules via an oxygen atom, an amino group, a sulfur atom, respectively.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃)alkoxy, particularly ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “cycloalkyl” refers to a mono cyclic or polycyclic non-aromatic radical, wherein each of the atoms forming the ring (i.e. skeletal atoms) is a carbon atom. In one embodiment, the cycloalkyl group is saturated or partially unsaturated. In another embodiment, the cycloalkyl group is fused with an aromatic ring. Cycloalkyl groups include groups having from 3 to 10 ring atoms. Illustrative examples of cycloalkyl groups include, but are not limited to, the following moieties:

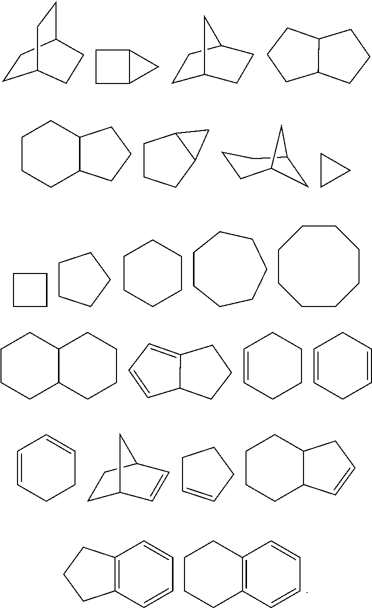

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon carbon double bond or one carbon carbon triple bond.

As used herein, the term “heteroalkyl” by itself or in combination with another term means, unless otherwise stated, a stable straight or branched chain alkyl group consisting of the stated number of carbon atoms and one or two heteroatoms selected from O, N, Si, P, or S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃,
—CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃.

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from N, O, or S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine. Other non-limiting examples of heterocycloalkyl groups are:

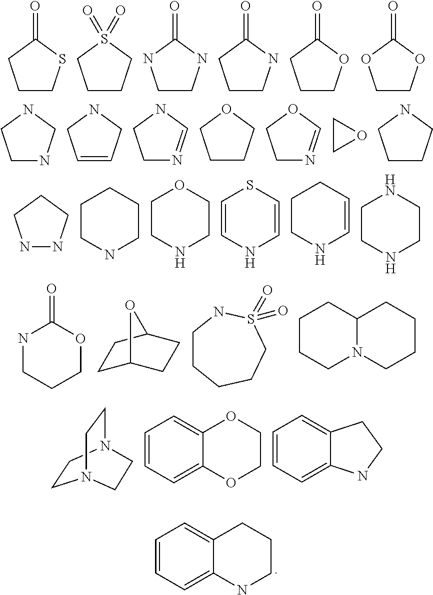

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

As used herein, the term “aromatic” refers to a carbocycle or heterocycle with one or more polyunsaturated rings and having aromatic character, i.e. having (4n+2) delocalized p (pi) electrons, where n is an integer.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. Preferred are phenyl and naphthyl, most preferred is phenyl.

As used herein, the term “aryl-(C₁-C₄)alkyl” means a functional group wherein a one to three carbon alkylene chain is attached to an aryl group, e.g., —CH₂CH₂-phenyl. Preferred is aryl-CH₂— and aryl-CH(CH₃)—. The term “substituted aryl-(C₁-C₄)alkyl” means an aryl-(C₁-C₄)alkyl functional group in which the aryl group is substituted. Preferred is substituted aryl (CH₂)—. Similarly, the term “heteroaryl-(C₁-C₄)alkyl” means a functional group wherein a one to three carbon alkylene chain is attached to a heteroaryl group, e.g., —CH₂CH₂-pyridyl. Preferred is heteroaryl-(CH₂)—. The term “substituted heteroaryl-(C₁-C₄)alkyl” means a heteroaryl-(C₁-C₄)alkyl functional group in which the heteroaryl group is substituted. Preferred is substituted heteroaryl-(CH₂)—.

Examples of heteroaryl groups include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “amino aryl” refers to an aryl moiety which contains an amino moiety. Such amino moieties may include, but are not limited to primary amines, secondary amines, tertiary amines, masked amines, or protected amines. Such tertiary amines, masked amines, or protected amines may be converted to primary amine or secondary amine moieties. Additionally, the amine moiety may include an amine-like moiety which has similar chemical characteristics as amine moieties, including but not limited to chemical reactivity.

As used herein, the term “substituted” means that an atom or group of atoms has replaced hydrogen as the substituent attached to another group. For aryl, aryl-(C₁-C₄)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two. In yet another embodiment, the substituents are independently selected from C₁₋₆ alkyl, —OH, C₁₋₆ alkoxy, halo, amino, acetamido, or nitro. In yet another embodiment, the substituents are independently selected from C₁₋₆ alkyl, C₁-6 alkoxy, halo, acetamido, or nitro. As used herein, where a substituent is an alkyl or alkoxy group, the carbon chain may be branched, straight or cyclic, with straight being preferred.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

In one embodiment, the substituents are independently selected from oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)₂alkyl, —C(═O) NH [substituted or unsubstituted alkyl, or substituted or unsubstituted phenyl], —C(═O)N[H or alkyl]₂, —OC(═O) N [substituted or unsubstituted alkyl]₂, —NHC(═O) NH [substituted or unsubstituted alkyl, or substituted or unsubstituted phenyl], —NHC(═O)alkyl, —N [substituted or unsubstituted alkyl] C(═O) [substituted or unsubstituted alkyl], —NHC(═O) [substituted or unsubstituted alkyl], —C(OH) [substituted or unsubstituted alkyl]₂, or —C(NH₂) [substituted or unsubstituted alkyl]₂. In another embodiment, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O) CH₃, —ON(O)₂, or —C(═O)OH. In yet one embodiment, the substituents are independently selected from C₁₋₆ alkyl, —OH, C₁₋₆ alkoxy, halo, amino, acetamido, oxo, or nitro. In yet another embodiment, the substituents are independently selected from C₁₋₆ alkyl, C₁₋₆ alkoxy, halo, acetamido, or nitro. As used herein, where a substituent is an alkyl or alkoxy group, the carbon chain may be branched, straight or cyclic.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

A “disease” is a state of health of an animal that is compromised by biologically initiated processes. For example, such biological processes may be pathogens, such as viruses and bacteria. They can also be genetic diseases or autoimmune diseases caused by attack by the immune system on the cells of their own bodies. They may also manifest as the body's reaction to a physiological insult, such as exposure to lethal levels of radiation.

The term “disorder” generally refers to any disturbance of normal functioning of the mind or body. For example, disorders are disruptions to normal health that are the subject of the current invention are not diseases, as defined in the above descriptions. The present invention is focused on disruptions to normal health and tissue function that are specifically caused by exposure to pathological insults that cause severe or critical hyper-activation of the immune system, and is directed at the prevention of death and severe morbidity induced by such pathological insults. These may include infectious pathogens, but also may include such other conditions of severe or critical changes in immune system function caused by, e.g., graft-versus-host disease, radiation exposure, such as an exposure to ionizing radiation or a lethal radiation exposure, sepsis, stress, such as a chronic stress, aging, Alzheimer's disease, concomitant infection, such as an infection with HIV, exposure to chemotherapeutic agents or other toxic insults, etc. In another aspect, the present invention is directed more generally at any insult in which development of excessive immune activation (e.g., hypercytokinemia, also known as a cytokine storm and as cytokine release syndrome) or immune system exhaustion can cause morbidity or mortality.

There is no single characteristic that describes such disruptions as they may vary in their details and nuances with different pathogens, different ages, different genders, different genetic backgrounds, different background diseases states, different diets, and multiple other influences well known to those skilled in the relevant art. As with all disorders, there are biological consequences. In the case of disruptions to normal health and tissue function caused by exposure to pathological insults that cause severe or critical hyper-activation of the immune system, or other severe or critical disruptions of immune system function, the present invention is directed at the prevention of death and severe morbidity induced by such pathological insults.

As used herein, the term “severity of a disease” refers to the risk posed by the disease to a subject. Severity of a disease also dictates the extent of treatment necessary for appropriately treating the subject. For example, a disease can be mild, moderate, severe, or critical.

A mild disease may cause slight discomfort and may resolve without any treatment, for example, where a subject's immune system neutralizes the disease. A moderate disease may cause more than slight discomfort and may require some treatment for the disease to resolve. A severe disease is qualitatively different from moderation or mild disease, and causes significant discomfort and would require extensive treatment, including interventions beyond those used in treating moderate disease. A critical disease is life threatening and would require hospitalization and extensive treatment, which may not be successful resulting in the subject's death.

An “infectious pathogen” is defined as an organism causing disease to its host, and that is communicable by infection, as from one person to another or from one part of the body to another. The severity of the disease symptoms referred to as virulence. Pathogens are taxonomically widely diverse and comprise viruses and bacteria as well as unicellular and multicellular eukaryotes.

“Acute respiratory distress syndrome (ARDS)” is a respiratory failure caused by rapid and widespread inflammation in the lungs. In ARDS, fluid builds up in the alveoli thereby preventing the lungs from filling with enough air and reduced oxygen supply to the organs. “Sepsis” is a potentially life-threatening condition caused by excessive inflammatory response to a pathogenic infection. The excessive inflammatory response can trigger changes that can damage multiple organ systems.

“Systemic inflammatory response syndrome (SIRS)” and “chronic inflammatory response syndrome (CIRS)” are widespread inflammatory states affecting the entire body. Unlike sepsis, which is in response to an infection, SIRS and CIRS can be in response to an infectious or noninfectious insult.

Toxic shock syndrome is a sudden and potentially fatal condition caused by the release of toxins from an infection, such as Staphylococcus aureus.

By “hypercytokinemia disease” is meant a disease condition that is characterized by the presence of hypercytokinemia (also known as a cytokine storm). As used herein, hypercytokinemia refers to a severe immune reaction in which the body releases too many cytokines into the blood too quickly. Cytokines play an important role in normal immune responses, but having a large amount of them released in the body all at once can be harmful. Hypercytokinemia can occur as a result of an infection, graft-versus-host disease, or other disease. It may also occur after treatment with some types of immune-therapy. Signs and symptoms include high fever, inflammation (redness and swelling), and severe fatigue and nausea. Sometimes, hypercytokinemia may be severe or life threatening and lead to multiple organ failure. In embodiments, hypercytokinemia diseases may be caused by a viral infection. For example, the hypercytokinemia disease that may be treated by methods of the invention occurs as a result of exposure to an infectious pathogen capable of causing severe or critical disruptions in normal tissue function, or to such other insults capable of compromising an immune system function (e.g., pathological insults capable of causing excess immune activation or immune exhaustion) as graft-versus-host disease, sepsis, and/or radiation exposure. In some instances, the disease is severe or critical.

The term “immune system exhaustion” or “immune exhaustion” refers to conditions in which the prolonged response to a pathogen causes a failure to respond effectively to the same or other infections. Immune system exhaustion is an example of the more general condition of a compromised immune response. Immune system exhaustion may occur for multiple reasons, such as T-cell anergy, depletion of memory B cells, or other changes that prevent an adequate response of the immune system to infectious agents.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

By the term “modulating,” as used herein, is meant mediating a detectable increase or decrease in the level of a tissue function or a response in a subject compared with the level of a tissue function or a response in the subject in the absence of a treatment or compound, and/or compared with the level of a tissue function or a response in an otherwise identical but untreated subject. The term encompasses perturbing and/or affecting a native signal or response thereby mediating a beneficial therapeutic response in a subject, preferably, a human.

A disease or disorder is “alleviated” if the severity of a sign or symptom of the disease or disorder, the frequency with which such a sign or symptom is experienced by a patient, or both, is reduced. The sign or symptom may range from morbidity to death.

To “treat” a disease or disorder as the term is used herein, means to reduce the frequency or severity of at least one sign or symptom of a disease or disorder experienced by a subject. It may also mean preventing death or long-term tissue dysfunction experienced by a subject as a result of exposure to a pathogen (e.g., an infectious pathogen) capable of causing severe or critical disruptions in normal tissue function, or to such other insults capable of compromising an immune system function (e.g., pathological insults capable of causing excess immune activation or immune exhaustion) as graft-versus-host disease, sepsis, and/or radiation exposure.

The term “treating” refers to the administration of a therapeutically effective amount of a therapeutic agent (e.g., 4-AP) to a subject known or suspected to be exposed to an infectious pathogen capable of causing severe or critical disruptions in normal tissue function, or to such other insults capable of compromising an immune system function (e.g., pathological insults capable of causing excess immune activation or immune exhaustion) as graft-versus-host disease, sepsis, and/or radiation exposure.

The term “treatment” refers to the medical management of a patient with the intent to cure, ameliorate, stabilize, or prevent a pathological condition, or disorder. This term includes active treatment, that is, treatment directed specifically toward the improvement of a pathological condition, or disorder. In general, it may include a causal treatment directed toward removal of the cause (e.g., the infectious pathogen(s)) of the associated pathological condition, or disorder. In contrast, treatment, as defined within this application, also includes preventative treatment, that is, treatment directed to minimizing or partially or completely inhibiting the development of damage that may be caused by exposure to an infectious pathogen capable of causing severe or critical disruptions in normal tissue function, or to such other insults capable of compromising an immune system function (e.g., pathological insults capable of causing excess immune activation or immune exhaustion) as graft-versus-host disease, sepsis, and/or radiation exposure. Such treatments may be employed to supplement another specific therapy directed toward the durable improvement of the associated disease, pathological condition, or disorder.

As used herein, “treatment of disruptions to normal health and tissue function that are specifically caused as a result of exposure to an infectious pathogen capable of causing severe or critical disruptions in normal tissue function, or to such other insults capable of compromising an immune system function (e.g., pathological insults capable of causing excess immune activation or immune exhaustion) as graft-versus-host disease, sepsis, and/or radiation exposure” means reducing the severity and/or frequency with which a sign or symptom of the disruption to normal health and tissue function is experienced by a subject. Such treatments may include prevention of the disruption, stabilization of the disruption to slow or prevent progressive deterioration, reversal of the disruption, and/or partial or complete restoration of normal tissue structure and/or function. Treatments may also include management of symptoms of the tissue disruption to reduce their severity, with the goal of preventing, stabilizing or reversing dysfunction, and/or partially or completely restoring normal tissue structure and/or function.

Thus, by “treatment” it is meant that at least an amelioration of one or more symptoms associated with the condition afflicting the subject is achieved, where amelioration is used in a broad sense to refer to at least a reduction in the magnitude of a parameter, e.g., a symptom associated with the impairment being treated. As such, treatment also includes situations where a pathological condition, or at least symptoms associated therewith, are completely inhibited, e.g., prevented from happening, or stopped, e.g., terminated, such that the adult mammal no longer suffers from the impairment, or at least from the symptoms that characterize the impairment. In some instances, “treatment”, “treating” and the like refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” may be any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. Treatment may result in a variety of different physical manifestations, e.g., rejuvenation of tissue or organs, changes in cytokine levels, etc. Treatment of ongoing disease, where the treatment stabilizes or reduces the undesirable clinical symptoms of the patient, occurs in some embodiments. The subject therapy may be administered prior to the symptomatic state of the disease, during the symptomatic stage of the disease, and in some cases after the symptomatic stage of the disease.

“Treatment for prognostic purposes” refers to the use of the methods of the invention in order to identify individuals likely to benefit from continued treatment. Such treatments for prognostic purposes generally employ short-term treatment, for example (but not limited to) between one and five days of treatment and analysis of any of the symptoms of tissue dysfunction caused by exposure to a pathogen or another inducer of immune exhaustion and/or excessive immune activation. In this way, responders to the treatment can be identified early so as to focus further attention on those individuals most likely to benefit from the treatments of the present invention.

As used herein, the terms “therapy” or “therapeutic regimen” refer to those activities taken to alleviate or alter a disruption, or disruptions, to normal health and tissue function that are specifically caused as a result of exposure to an infectious pathogen capable of causing severe or critical disruptions in normal tissue function, or to such other insults capable of compromising an immune system function (e.g., pathological insults capable of causing excess immune activation or immune exhaustion) as graft-versus-host disease, sepsis, and/or radiation exposure, e.g., a course of treatment intended to reduce or eliminate at least one sign or symptom of such disruptions using pharmacological, surgical, dietary and/or other techniques. In general, a therapeutic regimen may include a prescribed dosage of one or more drugs or surgery. In the case of the present invention, a therapeutic regimen may consist of treatment with 4-AP, a derivative thereof, or a combination of such agents. Such regimens may include other agents, such as anticonvulsants, to prevent possible side effects of 4-AP dosages in individuals of increased susceptibility to induction of seizures (which are not caused by dosages generally used in treatments with these agents). Therapies will most often be beneficial and reduce or eliminate at least one sign or symptom of the disruption of normal health and tissue function. The effect of therapy will also be impacted by the physiological state of the subject, e.g., age, gender, genetics, weight, other disease conditions, etc.

A “therapeutic” treatment is a treatment administered to a subject who exhibits signs of pathology, for the purpose of diminishing or eliminating those signs and/or preventing death.

The term “nephropathy” refers to the broad category of situations in which renal function is compromised in some way. The term nephropathy is used broadly to describe the symptomatic result of a variety of aspects of renal dysfunction, with no inferences as to the cause of such dysfunctions. Such tissue dysfunction may occur, for example, as a result of exposure to an infectious pathogen capable of causing severe or critical disruptions in normal tissue function, or to such other insults capable of compromising an immune system function (e.g., pathological insults capable of causing excess immune activation or immune exhaustion) as graft-versus-host disease, sepsis, and/or radiation exposure, which may differ from nephropathies induced by such causes as kidney disease or ischemia-reperfusion injuries to a variety of different tissues, as may occur for example in myocardial infarctions. Nephropathies caused by exposure to an infectious pathogen capable of causing severe or critical disruptions in normal tissue function, or to such other insults capable of compromising an immune system function (e.g., pathological insults capable of causing excess immune activation or immune exhaustion) as graft-versus-host disease, sepsis, and/or radiation exposure represent an important unmet medical need.

One example of damage that may be caused as a result of exposure to an infectious pathogen capable of causing severe or critical disruptions in normal tissue function (including, but not limited to certain viral or bacterial infections, including Lyme disease, shingles, hepatitis B, hepatitis C, leprosy, diphtheria, and/or HIV), or to such other insults capable of compromising an immune system function (e.g., pathological insults capable of causing excess immune activation or immune exhaustion) as graft-versus-host disease, sepsis, radiation exposure, such as an exposure to ionizing radiation or a lethal radiation exposure, and/or exposure to chemotherapeutic agents or other toxic insults, is a collection of conditions that are included in the broad grouping of “peripheral neuropathies” (as discussed for Covid-19 in Sasan Andalib et all, (2021) Peripheral Nervous System Manifestations Associated with COVID-19. Current Neurology and Neuroscience Reports 21:9). Peripheral neuropathies are a general description of a broad class of changes identified by symptoms presented, such as changes in sensation in peripheral nerves, but the underlying causes and mechanisms vary over a broad range. Thus, the term neuropathy describes symptoms, but includes a wide variety of different individual disruptions of normal tissue function. Thus, even though expression of some of the shared symptoms of peripheral neuropathies occurs in many different situations, there is little or no reason to believe that the underlying causes and pathologies are the same even for neuropathies caused by biological afflictions, such as diabetic neuropathy, neuropathic pain associated with spinal stenosis, peripheral neuropathy in autoimmune diseases, such as Guillain Barre Syndrome, neuropathic pain following spinal cord injury or stroke. The underlying causes and pathologies for neuropathies caused as a result of exposure to an infectious pathogen capable of causing severe or critical disruptions in normal tissue function, or to such other insults capable of compromising an immune system function (e.g., pathological insults capable of causing excess immune activation or immune exhaustion) as graft-versus-host disease, sepsis, and/or radiation exposure, may represent still a different broad category of afflictions that are united by the sharing of a symptom rather than by being caused by shared mechanisms or being treatable by shared approaches. Thus, it is understood that the term “peripheral neuropathy” is used only to indicate that a person expresses symptoms that would lead to their inclusion in this broad and multi-membered category, but that this term is not associated with specific causes or types of damage. There are many types of insults that can lead to outcomes that are collectively referred to as neuropathies, although that does not mean that the neuropathies are the same in respect to their detailed nature, their pathogenesis or their treatment.

Ataxias are other types of nervous system dysfunction that can be caused by infectious pathogens. These disorders of motor function go by many different names and causes, some of which are associated with known genetic mutations (and may be heritable) and some of which are of unknown etiology. They may manifest as gait ataxias, involuntary eye movements (nystagmus), incoordination of hands, slurring of speech, difficulty with fine motor tasks, and/or an unsteady walk. Ataxias can be co-morbidities of a diverse range of pathogens, such as chickenpox, HIV, and lyme disease. Many of the ataxias are referred to as cerebellar ataxias due to the importance of the cerebellum in motor function. Cerebellar ataxias may emerge as a result of infection with covid-19 or other viruses (e.g., influenza, Coxsackie disease, Epstein-Barr virus, etc.). Indeed, in children, acute cerebellar ataxia is more frequently caused by infection with a bacteria or a virus.

Similar concerns as stated in the above three paragraphs apply to all disruptions of normal tissue function, regardless of the tissue in which they occur, which means that applying a particular category designation to a dysfunction may or may not reveal information on either specific mechanisms of causation or remedy. As nonlimiting examples, dysfunctions of the visual system, auditory system, olfactory system, respiratory system, gastrointestinal system, genitourinary system, musculoskeletal system, peripheral nervous system, central nervous system, musculoskeletal system, and other parts of the body may be caused by many different means, and saying that a particular type of dysfunction exists in a particular tissue generally reveals no information on either mechanisms of causation or remedy.

Thus, the observations that tissue dysfunction caused as a result of exposure to an infectious pathogen capable of causing severe or critical disruptions in normal tissue function, or to such other insults capable of compromising an immune system function (e.g., pathological insults capable of causing excess immune activation or immune exhaustion) as graft-versus-host disease, sepsis, radiation exposure, such as an exposure to ionizing radiation or a lethal radiation exposure, concomitant infection, such as an infection with HIV, and/or exposure to chemotherapeutic agents or other toxic insults, may have characteristics that overlap with tissue dysfunction caused in other ways means that, as a general principle, the use of a similar term to place outcomes in a certain functional category has no implications as to cause, pathological underpinnings or treatment of the tissue dysfunction. Nonetheless, the presence of a shared biological dysfunction thought to be important in the disease process may offer an opportunity to provide benefit by ameliorating that shared biological dysfunction.

An “effective amount” or “pharmaceutically effective amount” of a compound is that amount of compound which is sufficient to provide a beneficial effect to the subject to which the compound is administered. An “effective amount” of a delivery vehicle is that amount sufficient to effectively bind or deliver a compound. The phrase “therapeutically effective amount,” as used herein, refers to an amount that is sufficient or effective to prevent or treat (delay or prevent the onset of, prevent the progression of, inhibit, decrease or reverse) a disruption to normal health and tissue function caused as a result of exposure to an infectious pathogen capable of causing severe or critical disruptions in normal tissue function, or to such other insults capable of compromising an immune system function (e.g., pathological insults capable of causing excess immune activation or immune exhaustion) as graft-versus-host disease, sepsis, and/or radiation exposure, including alleviating symptoms of such disruptions.

A “therapeutically effective amount” refers to that amount which provides a therapeutic effect for a given condition and administration regimen. In particular, “therapeutically effective amount” means an amount that is effective to prevent, alleviate or ameliorate symptoms of the disruption to normal health and/or tissue function that are caused as a result of exposure to an infectious pathogen capable of causing severe or critical disruptions in normal tissue function, or to such other insults capable of compromising an immune system function (e.g., pathological insults capable of causing excess immune activation or immune exhaustion) as graft-versus-host disease, sepsis, and/or radiation exposure, or prolong the survival of the subject being treated, which may be a human or non-human animal. Determination of a therapeutically effective amount is within the skill of the person skilled in the art. A “therapeutically effective amount” refers to that amount of a therapeutic agent that will have a durable beneficial effect, which may be curative, on the health and well-being of the subject with regard to a disruption to normal health and tissue function caused as a result of exposure to an infectious pathogen capable of causing severe or critical disruptions in normal tissue function, or to such other insults capable of compromising an immune system function (e.g., pathological insults capable of causing excess immune activation or immune exhaustion) as graft-versus-host disease, sepsis, and/or radiation exposure. The beneficial effect on the health and well-being of a subject can include, but is not limited to: (1) curing the condition; (2) slowing the progress of the condition; (3) causing the condition to retrogress; (4) preventing death; (5) decreasing the symptoms caused as a result of exposure to an infectious pathogen capable of causing severe or critical disruptions in normal tissue function, or to such other insults capable of compromising an immune system function (e.g., pathological insults capable of causing excess immune activation or immune exhaustion) as graft-versus-host disease, sepsis, and/or radiation exposure. The beneficial effect on the health and well-being of a subject can also include prophylactic outcomes, including but not limited to: (1) preventing or delaying on-set of the damage to at least one tissue as a result of exposure to an infectious pathogen capable of causing severe or critical disruptions in normal tissue function, or to such other insults capable of compromising an immune system function (e.g., pathological insults capable of causing excess immune activation or immune exhaustion) as graft-versus-host disease, sepsis, and/or radiation exposure; (2) maintaining the damage at a retrogressed level once such level has been achieved by a therapeutically effective amount of a substance; (3) preventing or delaying recurrence of the damage after a course of treatment; or, (4) decreasing the likelihood of tissue damage as a result of exposure to an infectious pathogen capable of causing severe or critical disruptions in normal tissue function, or to such other insults capable of compromising an immune system function (e.g., pathological insults capable of causing excess immune activation or immune exhaustion) as graft-versus-host disease sepsis, and/or radiation exposure, or (5) decreasing any part of the symptoms caused as a result of exposure to an infectious pathogen capable of causing severe or critical disruptions in normal tissue function, or to such other insults capable of compromising an immune system function (e.g., pathological insults capable of causing excess immune activation or immune exhaustion) as graft-versus-host disease, sepsis, and/or radiation exposure.

The term “pharmacological composition,” “therapeutic composition,” “therapeutic formulation” or “pharmaceutically acceptable formulation” can mean, but is in no way limited to, a composition or formulation that allows for the effective distribution of an agent provided by the invention, which is in a form suitable for administration to the physical location most suitable for their desired activity, e.g., systemic administration. As used herein, the term “pharmaceutical composition” refers to a mixture of at least one compound of the invention with other chemical components and entities, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, topical, intraperitoneal, intramuscular, oral, aerosol, parenteral, ophthalmic, pulmonary, and topical administration. Non-limiting examples of agents suitable for formulation with the, e.g., compounds provided by the instant invention include: cinnamoyl, PEG, phospholipids or lipophilic moieties, phosphorothioates, P-glycoprotein inhibitors (such as Pluronic P85) which can enhance entry of drugs into various tissues, for example the CNS (Jolliet-Riant and Tillement, 1999, Fundam. Clin. Pharmacol., 13, 16-26); biodegradable polymers, such as poly (DL-lactide-coglycolide) microspheres for sustained release delivery after implantation (Emerich, D F et al, 1999, Cell Transplant, 8, 47-58) Alkermes, Inc. Cambridge, Mass.; loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999); and oil-based delivery systems (Kirtane et al. 2022, Sci. Adv. 8, eabm8478).

The term “pharmaceutically acceptable” or “pharmacologically acceptable” can mean, but is in no way limited to, entities and compositions that do not produce an adverse, allergic or other untoward reaction when administered to an animal, or a human, as appropriate. “Pharmaceutically acceptable”, as used herein, refers to compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the Food and Drug Administration.

The term “pharmaceutically acceptable salt” refers to any pharmaceutically acceptable salt, which upon administration to the subject is capable of providing (directly or indirectly) a compound as described herein. Such salts preferably are acid addition salts with physiologically acceptable organic or inorganic acids. Examples of the acid addition salts include mineral acid addition salts such as, for example, hydrochloride, hydrobromide, hydroiodide, sulphate, nitrate, phosphate, and organic acid addition salts such as, for example, acetate, trifluoroacetate, maleate, fumarate, citrate, oxalate, succinate, tartrate, malate, mandelate, methane sulphonate, and p-toluenesulphonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine, and basic amino acids salts. However, it will be appreciated that non-pharmaceutically acceptable salts also fall within the scope of the invention since those may be useful in the preparation of pharmaceutically acceptable salts. Procedures for salt formation are conventional in the art.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; surface active agents; alginic acid; pyrogen-free water, isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art.

“Pharmaceutically acceptable excipient” refers to an excipient that is conventionally useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

The term “solvate” in accordance with the present invention should be understood as meaning any form of the active compound in accordance with the invention in which the said compound is bonded by a non-covalent bond to another molecule (normally a polar solvent), including especially hydrates and alcoholates.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intradermal, intrasternal injection, intratumoral, intravenous, intracerebroventricular and kidney dialytic infusion techniques.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention is based, in part, on the unexpected results that 4-aminopyridine (4-AP) effectively prevented death and morbidity induced by an exposure to an infectious pathogen capable of causing severe or critical disruptions in a normal tissue function. Thus, the present invention relates, in part, compositions and methods for treating a subject exposed to an infectious pathogen capable of causing severe or critical disruptions in normal tissue function, or to such other insults capable of compromising an immune system function (e.g., pathological insults capable of causing excess immune activation or immune exhaustion) as graft-versus-host disease, radiation exposure, such as a lethal or near-lethal radiation exposure, sepsis, and/or other conditions that cause disruptions in normal immune system function that share similarities with the immune system dysfunction that occurs with exposure to an uncontrolled pathogen infection. The method can include administering to the subject a pharmaceutical composition comprising a potassium channel blocker. In some embodiments, the damage caused by the pathogen or different inducer of immune exhaustion and/or excessive immune activation can be multi-site and/or multi-organ.

In one aspect, the present invention is directed to a method of preventing death and severe morbidity induced by pathogens. In another aspect, the present invention is directed more generally at any insult in which development of excessive immune activation (for example, hypercytokinemia, also known as a cytokine storm and as cytokine release syndrome) is thought to be a major contributor to lethality or morbidity. Such excessive immune function can occur with uncontrolled pathogen infections, but also may be seen in graft-versus-host disease, sepsis, responses to radiation (e.g., ionizing radiation, lethal radiation, etc.), and various other physiological insults. The medical community is lacking treatments for physiological insults that cause immune activation excessive enough to potentially be lethal. The present invention provides compositions and methods of preventing death, or preventing, alleviating, or treating a subject suffering from infection with pathogens, characterized by frequent mortality and morbidity, and may be associated with excessive immune activation and/or hypercytokinemia. Thus, in one aspect, the present invention is directed at the prevention of death and severe morbidity induced by pathogens. In another aspect, the present invention is directed more generally at any insult in which development of excessive immune activation (for example, hypercytokinemia, also known as a cytokine storm and as cytokine release syndrome) is thought to be a major contributor to lethality or morbidity. The present invention also provides compositions and methods of preventing, alleviating, or treating a tissue damage caused by exposure to a toxic agent or restoring at least a portion of normal tissue function. In some embodiments, the methods include administering to the subject a therapeutically effective amount of a pharmaceutical composition comprising at least one potassium channel blocker. In some embodiments, the potassium channel blocker comprises 4-AP, a derivative thereof, or a combination thereof. In some embodiments, the pharmaceutical composition can be administered with an additional therapeutic agent, such as an anticonvulsant.

The medical community is also lacking in treatments for pathogen infections in individuals in which the immune system is compromised. The immune system can be compromised by various factors, such as genetic mutation, medical treatment (e.g., chemotherapy), exposure radiation (e.g., ionizing radiation, lethal radiation, etc.), stress, aging, Alzheimer's disease, and other causes. As shown in the below examples of benefits of 4-AP treatment in ATM KO mice, which provided a model for several different types of immune defects relevant to protection from pathogen infections, treatment with 4-AP was effective at protecting against influenza-induced morbidity and mortality. Thus, another embodiment of the present invention is the use of 4-AP treatment to prevent morbidity or mortality in an individual whose immune system is compromised by causes other than a pathogen.

In some embodiments, the method can include administering to the subject a therapeutically effective amount of a pharmaceutical composition comprising 4-AP, a derivative thereof, or a combination thereof. In some embodiments, the pharmaceutical composition can be formulated to provide sustained release of the 4-AP, derivative of 4-AP, or a combination thereof. In certain embodiments, 4-AP or a derivative thereof can be represented by a structure according to Formula (I)

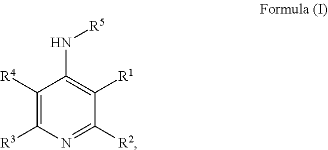

or an analog, a racemate, a tautomer, an isomer, an enantiomer, a diastereomer, a prodrug, a pharmaceutically acceptable salt, or a derivative thereof. In some embodiments, the derivative of 4-AP comprises 3,4-diaminopyridine, 3-hydroxy-4-aminopyridine, 3-methyl-4-aminopyridine, or a combination thereof.

In some embodiments, the methods described herein can be used for treating damage in a subject caused as a result of exposure to an infectious pathogen capable of causing severe or critical disruptions in normal tissue function, or to such other insults capable of compromising an immune system function (e.g., pathological insults capable of causing excess immune activation or immune exhaustion) as graft-versus-host disease, sepsis, and/or radiation exposure.

For example, the methods described herein can be used for treating the effects of infection with viral, bacterial or fungal pathogens that cause severe or critical hyper-activation of the immune system. In some embodiments, the methods described herein can be used for treating such immune system dysfunction caused by graft-versus-host disease. In some embodiments, the methods described herein can be used to restore or replenish immune function in an individual in need thereof (e.g., an individual suffering from immune exhaustion). In some embodiments, the methods described herein can be used to ameliorate morbidity or mortality of infections in an individual in need thereof (e.g., an individual with defects in immune system function).

In some embodiments, the damage can be most notable in one system of the body, such as in respiratory infections. In other embodiments, the damage may be manifested in more than one tissue, including, for example, the peripheral nervous system, the central nervous system, the visual system, the auditory system, the hematopoietic system, the gastrointestinal system, the bladder, the genitourinary system, the heart, the lungs, skeletal muscle, hair follicles, skin, vasculature, the salivary gland or a combination of any of these tissues.

In some embodiments, the methods described herein can restore at least a portion of decreased tissue function in the subject, enhance repair and regeneration of damaged tissue, enhance cell survival, reduce scarring, or combinations thereof, as compared to an untreated subject. In additional embodiments, the methods described herein can restore at least a portion of function of the damaged tissue, enhance repair and regeneration, enhance cell survival, reduce scarring, decrease other aspects of tissue damage or combinations thereof, as compared to an untreated subject.

In some embodiments, the methods disclosed herein can be used for preventing or treating muscle atrophy. In some embodiments, the muscle atrophy may be caused by influenza infection and particularly so in older individuals (Bartley et al., 2016, Aging 8:620-635). In some embodiments, the muscle atrophy may be caused by an exposure to an infectious pathogen capable of causing severe or critical disruptions in normal tissue function, or to such other insults capable of compromising an immune system function (e.g., pathological insults capable of causing immune system hyper-activation) as graft-versus-host disease, sepsis, and/or radiation exposure.

In other embodiments, the methods disclosed herein can be used for preventing or treating dysfunction in the peripheral nervous system, the central nervous system, the visual system, the auditory system, the hematopoietic system, the immune system, the gastrointestinal system, the bladder, the genitourinary system, the heart, lungs, skeletal muscle, hair follicles, skin, vasculature, the salivary gland, or any combination thereof. In some embodiments, the tissue dysfunction can be a result of exposure to an infectious pathogen capable of causing severe or critical disruptions in normal tissue function, or to such other insults capable of compromising an immune system function (e.g., pathological insults capable of causing immune system hyper-activation) as graft-versus-host disease, sepsis, and/or radiation exposure.

### Compounds and Compositions

In one aspect, the present invention provides compounds effective in preventing, alleviating, and/or treating a tissue damage, mitochondrial dysfunction, muscle atrophy, nephropathy or other types of dysfunctions in any other aspect of the body, or any combination thereof, as induced by an exposure to an infectious pathogen capable of causing severe or critical disruptions in a normal tissue function, or to such other insults capable of compromising an immune system function (e.g., pathological insults capable of causing excess immune activation or immune exhaustion) as graft-versus-host disease, sepsis, and/or radiation exposure. As nonlimiting examples, such dysfunctions may include dysfunctions of the visual system, auditory system, olfactory system, respiratory system, gastrointestinal system, genitourinary system, musculoskeletal system, peripheral nervous system, central nervous system, musculoskeletal system, and other parts of the body may be caused by many different means, and saying that a particular type dysfunction exists in particular tissue.

In one aspect, the present invention provides compounds effective in reducing or reversing a tissue damage caused by exposure to an infectious pathogen capable of causing severe or critical disruptions in normal tissue function, or to such other insults capable of compromising an immune system function (e.g., pathological insults capable of causing excess immune activation or immune exhaustion) including, but not limited to, a graft-versus-host disease, radiation exposure, such as an exposure to ionizing radiation or a lethal radiation exposure, sepsis, exposure to chemotherapeutic agents or other toxic insults, sepsis, and/or oxidation damage caused by such insults. In one aspect, the present invention provides compounds effective in restoring, improving, and/or enhancing at least a portion of tissue function effected by exposure to an infectious pathogen capable of causing severe or critical disruptions in normal tissue function, or to such other insults, such as pathological insults, that cause altered immune responses as graft-versus-host disease, sepsis, radiation, etc., and/or where the tissue function that is restored, improved, or enhanced includes, but is not limited to, preservation of myelin, replacement of myelin, tissue regeneration, cell survival, cell generation, repair or regeneration of endogenous stem or precursor cells, repair or regeneration of transplanted stem or precursor cells, repair or regeneration of stem or progenitor cells, or any combination thereof. In one aspect, the present invention provides compounds effective in reducing the lesion size caused as a result of exposure to an infectious pathogen capable of causing severe or critical disruptions in normal tissue function, or to such other insults, such as pathological insults, that cause immune hyper-activation as graft-versus-host disease, sepsis, and/or radiation exposure. In one aspect, the present invention provides compounds effective in inhibiting at least one ion channel that is affected by 4-AP or a derivative thereof.

Thus, in some embodiments, the compound is a potassium channel blocker or an analog, a racemate, a tautomer, an isomer, an enantiomer, a diastereomer, a prodrug, a pharmaceutically acceptable salt, or a derivative thereof having the structure of Formula (I)

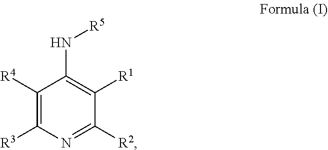

or an analog, a racemate, a tautomer, an isomer, an enantiomer, a diastereomer, a prodrug, a pharmaceutically acceptable salt, or a derivative thereof.

In various embodiments, R¹ is hydrogen, deuterium, halogen, alkyl (e.g., methyl, ethyl, propyl, butyl, pentyl, hexyl), haloalkyl (e.g., trifluoromethyl), cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy (e.g., methoxy, ethoxy, propoxy, butoxy, pentoxy, hexoxy), carboxyl, carboxylate, ester, ═O, —NO₂, —CN, sulfoxy, sulfonyl, alkyl sulfonyl, secondary amide, tertiary amide, an amino acid, or any combinations thereof. In some embodiments, R¹ is optionally substituted. For example, in some embodiments, R¹ is hydrogen, halogen, C₁-C₆ alkyl (e.g., methyl, ethyl, propyl, butyl, pentyl, hexyl), amine, hydroxyl, alkoxy (e.g., methoxy, ethoxy, propoxy, butoxy, pentoxy, hexoxy), carboxyl, or any combination thereof. In one embodiment, C₁-C₆ alkyl is a C₁-C₆ haloalkyl (e.g., trifluoromethyl). In some embodiments, R¹ is hydrogen, halogen, amine, hydroxyl, carboxyl, methyl, ethyl, propyl, butyl, pentyl, hexyl, methoxy, ethoxy, propoxy, butoxy, pentoxy, hexoxy, trifluoromethyl, or any combination thereof. In some embodiments, R¹ is hydrogen, halogen, amine, hydroxyl, carboxyl, methyl, methoxy, trifluoromethyl, or any combination thereof.

In various embodiments, R² is hydrogen, deuterium, halogen, alkyl (e.g., methyl, ethyl, propyl, butyl, pentyl, hexyl), haloalkyl (e.g., trifluoromethyl), cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy (e.g., methoxy, ethoxy, propoxy, butoxy, pentoxy, hexoxy), carboxyl, carboxylate, ester, ═O, —NO₂, —CN, sulfoxy, sulfonyl, alkyl sulfonyl, secondary amide, tertiary amide, an amino acid, or any combinations thereof. In some embodiments, R² is optionally substituted. For example, in some embodiments, R² is hydrogen, halogen, C₁-C₆ alkyl (e.g., methyl, ethyl, propyl, butyl, pentyl, hexyl), amine, hydroxyl, alkoxy (e.g., methoxy, ethoxy, propoxy, butoxy, pentoxy, hexoxy), carboxyl, or any combination thereof. In one embodiment, C₁-C₆ alkyl is a C₁-C₆ haloalkyl (e.g., trifluoromethyl). In some embodiments, R² is hydrogen, halogen, amine, hydroxyl, carboxyl, methyl, ethyl, propyl, butyl, pentyl, hexyl, methoxy, ethoxy, propoxy, butoxy, pentoxy, hexoxy, trifluoromethyl, or any combination thereof. In some embodiments, R² is hydrogen, halogen, amine, hydroxyl, carboxyl, methyl, methoxy, trifluoromethyl, or any combination thereof.

In various embodiments, R³ is hydrogen, deuterium, halogen, alkyl (e.g., methyl, ethyl, propyl, butyl, pentyl, hexyl), haloalkyl (e.g., trifluoromethyl), cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy (e.g., methoxy, ethoxy, propoxy, butoxy, pentoxy, hexoxy), carboxyl, carboxylate, ester, ═O, —NO₂, —CN, sulfoxy, sulfonyl, alkyl sulfonyl, secondary amide, tertiary amide, an amino acid, or any combinations thereof. In some embodiments, R³ is optionally substituted. For example, in some embodiments, R³ is hydrogen, halogen, C₁-C₆ alkyl (e.g., methyl, ethyl, propyl, butyl, pentyl, hexyl), amine, hydroxyl, alkoxy (e.g., methoxy, ethoxy, propoxy, butoxy, pentoxy, hexoxy), carboxyl, or any combination thereof. In one embodiment, C₁-C₆ alkyl is a C₁-C₆ haloalkyl (e.g., trifluoromethyl). In some embodiments, R³ is hydrogen, halogen, amine, hydroxyl, carboxyl, methyl, ethyl, propyl, butyl, pentyl, hexyl, methoxy, ethoxy, propoxy, butoxy, pentoxy, hexoxy, trifluoromethyl, or any combination thereof. In some embodiments, R³ is hydrogen, halogen, amine, hydroxyl, carboxyl, methyl, methoxy, trifluoromethyl, or any combination thereof.

In various embodiments, R⁴ is hydrogen, deuterium, halogen, alkyl (e.g., methyl, ethyl, propyl, butyl, pentyl, hexyl), haloalkyl (e.g., trifluoromethyl), cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy (e.g., methoxy, ethoxy, propoxy, butoxy, pentoxy, hexoxy), carboxyl, carboxylate, ester, ═O, —NO₂, —CN, sulfoxy, sulfonyl, alkyl sulfonyl, secondary amide, tertiary amide, an amino acid, or any combinations thereof. In some embodiments, R⁴ is optionally substituted. For example, in some embodiments, R⁴ is hydrogen, halogen, C₁-C₆ alkyl (e.g., methyl, ethyl, propyl, butyl, pentyl, hexyl), amine, hydroxyl, alkoxy (e.g., methoxy, ethoxy, propoxy, butoxy, pentoxy, hexoxy), carboxyl, or any combination thereof. In one embodiment, C₁-C₆ alkyl is a C₁-C₆ haloalkyl (e.g., trifluoromethyl). In some embodiments, R⁴ is hydrogen, halogen, amine, hydroxyl, carboxyl, methyl, ethyl, propyl, butyl, pentyl, hexyl, methoxy, ethoxy, propoxy, butoxy, pentoxy, hexoxy, trifluoromethyl, or any combination thereof. In some embodiments, R⁴ is hydrogen, halogen, amine, hydroxyl, carboxyl, methyl, methoxy, trifluoromethyl, or any combination thereof.

In various embodiments, R⁵ is hydrogen, deuterium, halogen, alkyl (e.g., methyl, ethyl, propyl, butyl, pentyl, hexyl), haloalkyl (e.g., trifluoromethyl), cycloalkyl, heterocycloalkyl, alkenyl, cycloalkenyl, alkynyl, cycloalkynyl, aryl, aryl alkyl, heteroaryl, heteroaryl alkyl, alkoxycarbonyl, amino, aminoalkyl, aminoaryl, amino alkyl-aryl, aminoheteroaryl, amino alkyl-heteroaryl, amido, aminoalkenyl, aminoalkynyl, aminoacetate, acyl, hydroxyl, hydroxyalkyl, hydroxyalkenyl, hydroxyalkynyl, hydroxyaryl, alkoxy (e.g., methoxy, ethoxy, propoxy, butoxy, pentoxy, hexoxy), carboxyl, carboxylate, ester, ═O, —NO₂, —CN, sulfoxy, sulfonyl, alkyl sulfonyl, secondary amide, tertiary amide, an amino acid, or any combinations thereof. In some embodiments, R⁵ is optionally substituted. For example, in some embodiments, R⁵ is hydrogen, halogen, C₁-C₆ alkyl (e.g., methyl, ethyl, propyl, butyl, pentyl, hexyl), amine, hydroxyl, alkoxy (e.g., methoxy, ethoxy, propoxy, butoxy, pentoxy, hexoxy), carboxyl, or any combination thereof. In one embodiment, C₁-C₆ alkyl is a C₁-C₆ haloalkyl (e.g., trifluoromethyl). In some embodiments, R⁵ is hydrogen, halogen, amine, hydroxyl, carboxyl, methyl, ethyl, propyl, butyl, pentyl, hexyl, methoxy, ethoxy, propoxy, butoxy, pentoxy, hexoxy, trifluoromethyl, or any combination thereof. In some embodiments, R⁵ is hydrogen, halogen, amine, hydroxyl, carboxyl, methyl, methoxy, trifluoromethyl, or any combination thereof.

For example, in one embodiment, the compound represented by Formula (I) is selected from 4-AP, or an analog, a racemate, a tautomer, an isomer, an enantiomer, a diastereomer, a prodrug, a pharmaceutically acceptable salt, or a derivative thereof, 3,4-diaminopyridine, or an analog, a racemate, a tautomer, an isomer, an enantiomer, a diastereomer, a prodrug, a pharmaceutically acceptable salt, or a derivative thereof, 3-hydroxy-4-aminopyridine, or an analog, a racemate, a tautomer, an isomer, an enantiomer, a diastereomer, a prodrug, a pharmaceutically acceptable salt, or a derivative thereof, 3-methyl-4-aminopyridine, or an analog, a racemate, a tautomer, an isomer, an enantiomer, a diastereomer, a prodrug, a pharmaceutically acceptable salt, or a derivative thereof, N-(4-pyridyl)-t-butyl carbamate, or an analog, a racemate, a tautomer, an isomer, an enantiomer, a diastereomer, a prodrug, a pharmaceutically acceptable salt, or a derivative thereof, N-(4-pyridyl)ethyl carbamate, or an analog, a racemate, a tautomer, an isomer, an enantiomer, a diastereomer, a prodrug, a pharmaceutically acceptable salt, or a derivative thereof, N-(4-pyridyl) methyl carbamate, or an analog, a racemate, a tautomer, an isomer, an enantiomer, a diastereomer, a prodrug, a pharmaceutically acceptable salt, or a derivative thereof, N-(4-pyridyl) isopropyl carbamate, or an analog, a racemate, a tautomer, an isomer, an enantiomer, a diastereomer, a prodrug, a pharmaceutically acceptable salt, or a derivative thereof, or any combination thereof.

The compounds described herein may form salts with acids or bases, and such salts are included in the present invention. The term “salts” embraces addition salts of free acids or free bases that are compounds of the invention.

In one aspect, the present invention relates, in part, to compositions comprising one or more compounds of the present invention. In some embodiments, the composition comprises one or more compounds having the structure of Formula (I), or an analog, a racemate, a tautomer, an isomer, an enantiomer, a diastereomer, a prodrug, a pharmaceutically acceptable salt, or a derivative thereof. In some embodiments, the composition is the pharmaceutical composition.

In one aspect, the present invention provides compositions effective in reducing or reversing a tissue damage or dysfunction caused as a result of exposure to an infectious pathogen capable of causing severe or critical disruptions in normal tissue function, or to such other insults, such as pathological insults, that cause similar immune hyper-activation or immune exhaustion as graft-versus-host disease, sepsis, radiation exposure, such as an exposure to ionizing radiation or a lethal radiation exposure, exposure to chemotherapeutic agents or other toxic insults, oxidation damage, and/or other condition caused by an infectious pathogen or another inducer of immune exhaustion and/or excessive immune activation. In one aspect, the present invention provides compositions effective in restoring, improving, and/or enhancing at least a portion of tissue function that is compromised as a result of exposure to an infectious pathogen capable of causing severe or critical disruptions in normal tissue function, or to such other insults, such as pathological insults, that cause immune hyper-activation or immune exhaustion as graft-versus-host disease, sepsis, radiation exposure, such as an exposure to ionizing radiation or a lethal radiation exposure, and/or exposure to chemotherapeutic agents or other toxic insults, with compromised tissue function affecting any of a number of physiological functions, including, but not limited to, myelination, tissue regeneration, cell survival, stem or progenitor cell generation, repair or regeneration of endogenous stem or precursor cells, repair or regeneration of transplanted stem or precursor cells, repair or regeneration of stem or progenitor cells, or other types of dysfunctions in any other aspect of the body, or any combination thereof. As non-limiting examples, such dysfunctions may include dysfunctions of the visual system, auditory system, olfactory system, respiratory system, gastrointestinal system, genitourinary system, musculoskeletal system, peripheral nervous system, central nervous system, hematopoietic system, immune system, and/or other parts of the body and may be caused by many different means. In one aspect, the present invention also provides compositions effective in reducing the lesion size caused as a result of exposure to an infectious pathogen capable of causing severe or critical disruptions in normal tissue function, or to such other insults, such as pathological insults, that cause immune hyper-activation or exhaustion as graft-versus-host disease, sepsis and/or radiation exposure.

In some embodiments, the composition further comprises one or more therapeutic agent. In some embodiments, the therapeutic agent is any potassium channel blocker known in the art. Examples of such potassium channel blockers include, but are not limited to, bretylium, clofilium, dalfampridine, dofetilide, E-4031, ebastine, gliclazide, ibutilide, nifekalant, sematilide, sotalol, sulfonylureas, tedisamil, or any combination thereof.

In some embodiments, the composition further comprises one or more anticonvulsant agents. Examples of such anticonvulsant agents include, but are not limited to, barbiturate, benzodiazepine, bromide, carbamate, carboxamide, fatty acid, fructose or a derivative thereof, γ-aminobutyric acid (GABA) or an analog thereof, hydantoin, oxazolidinedione, proprionate, pyrimidinedione, pyrrolidine, succinimide, sulfonamide, triazine, urea, valproylamide, or any combination thereof.

In certain embodiments, the disease involving excess immune activation with or without hypercytokinemia further comprises an overproduction of immune cells and/or pro-inflammatory cytokines into the lungs of the subject. Hypercytokinemia may result in excessive inflammatory response in the lungs, which typically occurs in the infections in the lungs or other organs. Such excessive inflammatory response in the lung includes infiltration into the lungs by immune cells as well as excessive secretion of proinflammatory cytokines in the lungs, which in turn attracts more immune cells into the lungs. Thus, hypercytokinemia can also involve positive-feedback loop between activated cells and released cytokines.

In certain embodiments, the composition provides methods for decreasing or preventing the manifestation of immune system exhaustion as a consequence of pathogen infection, graft-versus-host disease, sepsis, radiation exposure, such as an exposure to ionizing radiation or a lethal radiation exposure, exposure to chemotherapeutic agents or other toxic insults, and/or other inducers of immune hyper-activation leading to immune exhaustion.

### Combinations

In one embodiment, the composition of the present invention comprises a combination of agents described herein. In certain embodiments, a composition comprising a combination of agents described herein has an additive effect, wherein the overall effect of the combination is approximately equal to the sum of the effects of each individual agent. In other embodiments, a composition comprising a combination of agents described herein has a synergistic effect, wherein the overall effect of the combination is greater than the sum of the effects of each individual agent.

A composition comprising a combination of agents comprises individual agents in any suitable ratio. For example, in one embodiment, the composition comprises a 1:1 ratio of two individual agents. However, the combination is not limited to any particular ratio. Rather any ratio that is shown to be effective is encompassed.

### Methods of Use

In one aspect, the invention provides methods of preventing, alleviating, and/or treating a tissue damage caused by exposure to a pathogen (e.g., an infectious pathogen) or another inducer of immune exhaustion and/or excessive immune activation capable of causing severe or critical disruptions in a normal tissue function, or to such other insults, such as pathological insults, that cause immune exhaustion and/or excessive immune activation as graft-versus-host disease, sepsis, radiation exposure, such as an exposure to ionizing radiation or a lethal radiation exposure, exposure to chemotherapeutic agents or other toxic insults, and/or condition where the tissue damage includes, but is not limited to, tissue dysfunction, mitochondrial dysfunction, muscle atrophy, neuropathy, nephropathy, or any combination thereof.

In one aspect, the present invention provides methods of reducing or reversing a tissue damage caused by exposure to a pathogen (e.g., an infectious pathogen) or another inducer of immune exhaustion and/or excessive immune activation capable of causing severe or critical disruptions in a normal tissue function, or to such other insults capable of compromising an immune system function (e.g., pathological insults capable of causing excess immune activation or immune exhaustion) as graft-versus-host disease, sepsis, radiation exposure, such as an exposure to ionizing radiation or a lethal radiation exposure, and/or exposure to chemotherapeutic agents or other toxic insults, including, but not limited to, oxidation damage and/or scarring.

In some embodiments, the tissue damage is a multi-tissue damage, multi-organ tissue damage, or any combination thereof. In some embodiments, the tissue damage is a kidney tissue damage, liver tissue damage, heart tissue damage, lung tissue damage, brain tissue damage, central nervous system damage, peripheral nerve tissue damage, peripheral neuropathy, nephropathy, neutropenia, gastrointestinal tract tissue damage, gut tissue damage, visual system tissue damage, auditory system tissue damage, skin tissue damage, bladder tissue damage, reproductive system tissue damage, hematopoietic system tissue damage, immune system damage, or any combination thereof.

In some embodiments, the tissue dysfunction is a motor dysfunction, sensory dysfunction, cognitive dysfunction, visual dysfunction, auditory dysfunction, kidney dysfunction, hematopoietic system dysfunction, respiratory system dysfunction, immune system dysfunction, normal skin function, salivary gland dysfunction, liver dysfunction, gall bladder dysfunction, gastrointestinal (GI) dysfunction, sexual dysfunction, or any combination thereof.

In one aspect, the present invention provides methods of restoring, improving, and/or enhancing at least a portion of tissue function effected by exposure to a pathogen (e.g., an infectious pathogen) or another inducer of immune exhaustion and/or excessive immune activation capable of causing severe or critical disruptions in a normal tissue function, or to such other insults capable of compromising an immune system function (e.g., pathological insults capable of causing excess immune activation or immune exhaustion) as graft-versus-host disease, sepsis, radiation exposure, such as an exposure to ionizing radiation or a lethal radiation exposure, exposure to chemotherapeutic agents or other toxic insults, and/or condition where tissue function to be restored includes, but is not limited to, myelination, tissue regeneration, cell survival, stem or progenitor cell generation, repair or regeneration of endogenous stem or progenitor cells, repair or regeneration of transplanted stem cells, repair or regeneration of progenitor cells, or any combination thereof.

In some embodiments, the tissue function is a motor function, sensory function, cognitive function, visual function, auditory function, kidney function, hematopoietic system function, immune system function, respiratory system function, normal skin function, salivary gland function, liver function, gall bladder function, gastrointestinal (GI) function, sexual function, or any combination thereof. In some embodiments, the tissue is a kidney tissue, liver tissue, heart tissue, lung tissue, brain tissue, central nervous system tissue, peripheral nerve tissue, gastrointestinal tract tissue, gut tissue, visual system tissue, auditory system tissue, skin tissue, bladder tissue, reproductive system tissue, hematopoietic system tissue, musculoskeletal tissue, or any combination thereof.

In one aspect, the present invention provides methods of reducing the lesion size effected by exposure to a pathogen (e.g., an infectious pathogen) or another inducer of immune exhaustion and/or excessive immune activation capable of causing severe or critical disruptions in a normal tissue function, or to such other insults, such as pathological insults, that cause immune hyper-activation or exhaustion as graft-versus-host disease, sepsis, radiation exposure, such as an exposure to ionizing radiation or a lethal radiation exposure, and/or exposure to chemotherapeutic agents or other toxic insults.

In one aspect, the present invention provides methods of inhibiting at least one potassium channel for the purpose of treating tissue damage caused by exposure to a pathogen (e.g., an infectious pathogen) or another inducer of immune exhaustion and/or excessive immune activation capable of causing severe or critical disruptions in a normal tissue function, such as graft-versus-host disease, sepsis, radiation exposure, such as an exposure to ionizing radiation or a lethal radiation exposure, and/or exposure to chemotherapeutic agents or other toxic insults.

In one aspect, the present invention provides methods of preventing, alleviating, or treating a hyper-activation of the immune system, hypercytokinemia, cytokine storm, cytokine release syndrome, immune system exhaustion, or any combination thereof in a subject in need thereof. In some embodiments, the hyper-activation of the immune system, hypercytokinemia, cytokine storm, cytokine release syndrome, immune system exhaustion, or any combination thereof is caused by a graft-versus-host disease or an exposure to a pathogen (e.g., an infectious pathogen) or another inducer of immune exhaustion and/or excessive immune activation capable of causing a severe or critical disruption in a normal tissue function.

In one aspect, the present invention provides methods of preventing, alleviating, or treating a graft-versus-host disease in a subject in need thereof.

In one aspect, the present invention provides methods of preventing, alleviating, or treating sepsis, which is typified by a severe immune response caused by pathogen infection that is not able to controlled in a subject in need thereof.

In one aspect, the present invention provides methods of preventing, alleviating, or treating the morbidity of mortality of a radiation (e.g., ionizing radiation, lethal radiation, etc.) exposure, which compromises normal immune function, in a subject in need thereof.

In some embodiments, the method comprises administering the treatment of the present invention to a subject with an immune function that is compromised by mutation, stress, injury, aging, Alzheimer's disease, exposure to a pathogen or another inducer of immune exhaustion and/or excessive immune activation, disease or treatments of a disease.

In additional embodiments, the method comprises administering the treatment of the present invention to an individual with mutations with the ATM gene, and in particular to an individual with ataxia telangiectasia. Such treatment may be used to prevent adverse sequelae of exposure to infectious pathogens that have the potential of accelerating progression of ataxia telangiectasia. Treatment may also be used to restore motor and/or sensory function after it has been lost as a consequence of disease progression.

In some embodiments, the method comprises administering the treatment of the present invention to an individual with ataxia. Such treatment may be used to prevent adverse sequelae of exposure to infectious pathogens that have the potential of accelerating progression and/or enhancing the severity of the ataxia. Treatment may also be used to restore motor and/or sensory function after it has been lost as a consequence of disease progression.

In some embodiments, the method comprises administering to the subject an effective amount of a composition comprising at least one compound (e.g., at least one compound of Formula (I)) or composition of the present invention.

In one aspect, the present invention provides methods comprising administering at least one compound of the present invention or a composition thereof to the subject, wherein the subject was exposed to and/or affected by exposure to a pathogen (e.g., an infectious pathogen) or another inducer of immune exhaustion and/or excessive immune activation capable of causing severe or critical disruptions in normal tissue function, or to such other insults capable of compromising an immune system function (e.g., pathological insults capable of causing excess immune activation or immune exhaustion) as a graft-versus-host disease, sepsis, radiation exposure, such as an exposure to ionizing radiation or a lethal radiation exposure, and/or exposure to chemotherapeutic agents or other toxic insults.

In some aspects, the present invention provides methods of administering an effective amount of any compound or pharmaceutical composition disclosed herein to the subject. Thus, in some aspects, the present invention also provides methods comprising administering an effective amount of any compound or pharmaceutical composition disclosed herein to the subject, wherein the subject was exposed to and/or affected by exposure to an infectious pathogen capable of causing severe or critical disruptions in normal tissue function, or to such other insults capable of compromising an immune system function (e.g., pathological insults capable of causing excess immune activation or immune exhaustion) as graft-versus-host disease, sepsis, radiation exposure, such as an exposure to ionizing radiation or a lethal radiation exposure, and/or exposure to chemotherapeutic agents or other toxic insults.

The composition of the invention may be administered to a patient or subject in need systematically, locally, or a combination thereof.

The composition of the invention may be administered to a patient or subject in need in a wide variety of ways, including by inhalation, such as aerosol inhalation, injection, ingestion, oral administration, transdermal administration, transfusion, implantation, sublingual administration, or transplantation. The compositions described herein may be administered to a patient subcutaneously, intradermally, intratumorally, intranodally, intramedullary, intramuscularly, intrathecally, intravenously (i.v.), or intraperitoneally. In one embodiment, the composition is administered systemically to the subject. In one embodiment, the compositions of the present invention are administered to a patient by i.v. injection. In one embodiment, the composition is administered locally to the subject. In one embodiment, the compositions of the present invention are administered to a patient topically. Any administration may be a single application of a composition of invention or multiple applications. Administrations may be to single site or to more than one site in the individual to be treated. Multiple administrations may occur essentially at the same time or separated in time.

Subjects to which administration of the pharmaceutical compositions of the invention is contemplated include, but are not limited to, humans and other primates, mammals including but not limited to non-human mammals such as non-human primates, cattle, pigs, horses, sheep, cats, and dogs.

Pharmaceutical compositions of the present invention may be administered in a manner appropriate to the disease to be treated (or prevented). The quantity and frequency of administration will be determined by such factors as the condition of the subject, and the type and severity of the subject's disease, although appropriate dosages may be determined by clinical trials.

When “therapeutic amount” is indicated, the precise amount of the compositions of the present invention to be administered can be determined by a physician with consideration of individual differences in age, weight, disease type, extent of disease, and condition of the patient (subject).

In practicing embodiments of the invention, active agent compositions may be administered according to any desired dosage, such as once per day, a few or several times per day, or even multiple times per day, depending upon, among other things, the indication being treated and the judgment of the prescribing physician. For example, in some instances, compositions that include one or more active agents may be administered once per day, a few or several times per day, or even multiple times per day, depending upon, among other things, the indication being treated and the judgment of the prescribing physician.

In those embodiments where an effective amount of an active agent is administered to the adult mammal, the amount or dosage is effective when administered for a suitable period of time so as to evidence a reduction in one or more symptoms of the target disease. In some instances, an effective amount or dose of active agent will not only slow or halt the progression of the disease condition but will also induce the reversal of the condition, i.e., will cause an improvement the subject's condition. Where desired, effectiveness of treatment may be assessed using any convenient protocol. Biochemically, by an “effective amount” or “effective dose” of active agent is meant an amount of active agent that will inhibit, antagonize, decrease, reduce, or suppress by about 20% or more, e.g., by 30% or more, by 40% or more, or by 50% or more, in some instances by 60% or more, by 70% or more, by 80% or more, or by 90% or more, in some cases by about 100%, i.e., to negligible amounts, and in some instances reverse, one or more target symptoms of the disease condition.

### Formulations/Pharmaceutical Compositions

The invention also encompasses the use of pharmaceutical compositions comprising a compound of the invention or a composition thereof. Such a pharmaceutical composition may comprise of at least one compound of the invention in a form suitable for administration to a subject, or the pharmaceutical composition may comprise at least one compound of the invention and one or more pharmaceutically acceptable carriers, one or more additional ingredients, or some combination of these. The compound of the invention may be present in the pharmaceutical composition in the form of a physiologically acceptable salt, such as in combination with a physiologically acceptable cation or anion, as is well known in the art.

Administration of the therapeutic agent in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated. The amount administered will vary depending on various factors including, but not limited to, the composition chosen, the particular disease, the weight, the physical condition, and the age of the subject, and whether prevention or treatment is to be achieved. Such factors can be readily determined by the clinician employing animal models or other test systems which are well known to the art.

Administration of the compositions of the invention in a method of treatment can be achieved in a number of different ways, using methods known in the art. In one embodiment, the method of the invention comprises systemic administration of the subject, including for example enteral or parenteral administration. In certain embodiments, the method comprises intradermal delivery of the composition. In another embodiment, the method comprises intravenous delivery of the composition. In some embodiments, the method comprises intramuscular delivery of the composition. In one embodiment, the method comprises subcutaneous delivery of the composition. In one embodiment, the method comprises inhalation of the composition. In one embodiment, the method comprises intranasal delivery of the composition.

Depending on the intended mode of administration, the pharmaceutical composition can be in the form of, for example, solids, semi-solids, liquids, solutions, suspensions (e.g., incorporated into microparticles, liposomes, etc.), emulsions, gels, or the like, preferably in unit dosage form suitable for single administration of a precise dosage. The pharmaceutical compositions can include, as noted above, an effective amount of the potassium channel blocker such as 4-AP, a derivative thereof, or a combination thereof, in combination with a pharmaceutically acceptable carrier and, in addition, can include other carriers, adjuvants, diluents, thickeners, buffers, preservatives, surfactants, etc. Pharmaceutical compositions can also include one or more additional active ingredients such as other medicinal agents, pharmaceutical agents, antimicrobial agents, anti-inflammatory agents, anesthetics, anti-convulsants, and the like.

The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

The therapeutic and prophylactic methods of the invention thus encompass the use of pharmaceutical compositions comprising at least one compound of the invention, to practice the methods of the invention. The pharmaceutical compositions useful for practicing the invention may be administered to deliver a dose of from 0.001 ng/kg/day and 100 mg/kg/day. For example, in some embodiments, the pharmaceutical compositions useful for practicing the invention may be administered to deliver a dose of from 0.005 mg/kg/day and 5 mg/kg/day. In one embodiment, the invention comprises administration of a dose which results in a concentration of the compound of the present invention from 10 nM and 10 μM in the serum of a mammal.

Typically, dosages which may be administered in a method of the invention to a mammal, preferably a human, range in amount from 0.01 μg to about 50 mg per kilogram of body weight of the mammal, while the precise dosage administered will vary depending upon any number of factors, including but not limited to, the type of mammal and type of disease state being treated, the age of the mammal and the route of administration and the specific agent or agents that are utilized. Preferably, the dosage of the compound will vary from about 0.1 μg to about 10 mg per kilogram of body weight of the mammal. More preferably, the dosage will vary from about 1 μg to about 5 mg per kilogram of body weight of the mammal. For example, in some embodiments, the dosage will vary from about 0.005 mg to about 5 mg per kilogram of body weight of the mammal.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

The pharmaceutical composition can contain from about 0.01 to about 99 percent of the potassium channel blocker (e.g., 4-AP or a derivative or analog thereof), together with the carriers and/or excipients. For example, the amount of potassium channel blocker (e.g., 4-AP, a derivative thereof, or a combination thereof), by weight of the pharmaceutical composition can be about 0.1% or greater, about 1% or greater, about 2% or greater, about 5% or greater, about 10% or greater, about 15% or greater, about 20% or greater, about 25% or greater, about 30% or greater, about 50% or greater, about 75% or greater, or about 90% or greater.

In some embodiments, the pharmaceutical composition is defined by its ability to achieve serum therapeutically effective concentrations of 4-AP or a derivative thereof. In some embodiments, such concentrations range from about 10 nM to about 1 μM 4-AP, or higher concentrations if combined with an anti-convulsant. Desired serum concentrations of 4-AP derivatives are defined by the ability of such agents to cause the desired therapeutic benefits without causing unacceptable side effects.

The pharmaceutical compositions described herein are used in a “therapeutically effective amount” of the potassium channel blocker (e.g., 4-AP, a derivative thereof, or a combination thereof). In some embodiments, the pharmaceutical composition can be formulated, such that when administered, it delivers a therapeutically effective amount of the potassium channel blocker (e.g., 4-AP, a derivative thereof, or a combination thereof) in an amount of 2.5 mg or greater. For example, the pharmaceutical composition when administered can deliver 3 mg or greater, 4 mg or greater, 5 mg or greater, 6 mg or greater, 7 mg or greater, 7.5 mg or greater, 8 mg or greater, 9 mg or greater, 10 mg or greater, 15 mg or greater, 20 mg or greater, 25 mg or greater, 30 mg or greater, 35 mg or greater, 40 mg or greater, 45 mg or greater, 50 mg or greater, 55 mg or greater, 60 mg or greater, 65 mg or greater, 70 mg or greater, 75 mg or greater, 80 mg or greater, 85 mg or greater, 90 mg or greater, or 95 mg or greater of potassium channel blocker (e.g., 4-AP, a derivative thereof, or a combination thereof).

The composition may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, and whether the administration is used to treat the dysfunction or to identify individuals for whom a full course of treatment is beneficial, etc.

In another aspect of the invention, the methods disclosed herein can be used to identify individuals who will benefit from a treatment with potassium channel blockers (e.g., 4-AP, a derivative of 4-AP, or any combination thereof). In some embodiments, individuals manifesting dysfunction in one or more tissues following an exposure to an infectious pathogen capable of causing severe or critical disruptions in normal tissue function, or to such other insults capable of compromising an immune system function (e.g., pathological insults capable of causing excess immune activation or immune exhaustion) as graft-versus-host disease, sepsis, radiation exposure, such as an exposure to ionizing radiation or a lethal radiation exposure, and/or exposure to chemotherapeutic agents or other toxic insults may be treated with the methods disclosed herein for between one and ten days to determine if treatment provides improvement in tissue function. Thus, in some embodiments, the methods disclosed herein can be used to provide personalized targeting of therapies.

When the therapeutic agents of the invention are prepared for administration, they are preferably combined with a pharmaceutically acceptable carrier, diluent, or excipient to form a pharmaceutical formulation, or unit dosage form. The total active ingredients in such formulations include from 0.1 to 99.9% by weight of the formulation. A “pharmaceutically acceptable” carrier, diluent, or excipient is a carrier, diluent, excipient, and/or salt that is compatible with the other ingredients of the formulation, and not deleterious to the recipient thereof. The active ingredient for administration may be present as a powder or as granules; as a solution, a suspension or an emulsion.

Pharmaceutical formulations containing the therapeutic agents of the invention can be prepared by procedures known in the art using well known and readily available ingredients. The therapeutic agents of the invention can also be formulated as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g., sterile pyrogen free water) prior to parenteral administration of the reconstituted composition.

It will be appreciated that the unit content of active ingredient or ingredients contained in an individual aerosol dose of each dosage form need not in itself constitute an effective amount for treating the particular indication or disease since the necessary effective amount can be reached by administration of multiple dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions, such as phosphate buffered saline solutions pH 7.0-8.0.

The compounds of the present invention can be formulated and administered to treat a variety of disease states by any means that produces contact of the active ingredient with the agent's site of action in the body of the organism. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

In general, water, suitable oil, saline, aqueous dextrose (glucose), and related sugar solutions and glycols such as propylene glycol or polyethylene glycols are suitable carriers for parenteral solutions. Solutions for parenteral administration contain the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium Ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

Additionally, standard pharmaceutical methods can be employed to control the duration of action. These are well known in the art and include control release preparations and can include appropriate macromolecules, for example polymers, polyesters, polyamino acids, polyvinyl, pyrrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly (lactic acid) or ethylenevinylacetate copolymers. In addition to being incorporated, these agents can also be used to trap the compound in microcapsules.

Accordingly, the pharmaceutical composition of the present invention may be delivered via various routes and to various sites in a mammal body to achieve a particular effect (see, e.g., Rosenfeld et al., 1991; Rosenfeld et al., 1991a; Jaffe et al., supra; Berkner, supra). One skilled in the art will recognize that although more than one route can be used for administration, a particular route can provide a more immediate and more effective reaction than another route. Local or systemic delivery can be accomplished by administration comprising application or instillation of the formulation into body cavities, inhalation or insufflation of an aerosol, or by parenteral introduction, comprising intramuscular, intravenous, peritoneal, subcutaneous, intradermal, as well as topical administration.

The active ingredients of the present invention can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and mammal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the pharmaceutical composition in the particular host.

In one embodiment, the compositions of the invention are formulated using one or more pharmaceutically acceptable excipients or carriers. In one embodiment, the pharmaceutical compositions of the invention comprise a therapeutically effective amount of a compound or conjugate of the invention and a pharmaceutically acceptable carrier. Pharmaceutically acceptable carriers that are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin. In one embodiment, the pharmaceutically acceptable carrier is not DMSO alone.

The present invention also provides pharmaceutical compositions comprising one or more of the compositions described herein. Formulations may be employed in admixtures with conventional excipients, i.e., pharmaceutically acceptable organic or inorganic carrier substances suitable for administration to subject. The pharmaceutical compositions may be sterilized and if desired mixed with auxiliary agents, e.g., lubricants, preservatives, stabilizers, wetting agents, emulsifiers, salts for influencing osmotic pressure buffers, coloring, and/or aromatic substances and the like. They may also be combined when desired with other active agents, e.g., analgesic agents or anti-convulsants.

As used herein, “additional ingredients” include, but are not limited to, one or more of the following: excipients; surface active agents; dispersing agents; inert diluents; granulating and disintegrating agents; binding agents; lubricating agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, PA), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from benzyl alcohol, sorbic acid, parabens, imidurea, or any combinations thereof. A particularly preferred preservative is a combination of about 0.5% to 2.0% benzyl alcohol and 0.05% to 0.5% sorbic acid.

In an embodiment, the composition includes an anti-oxidant and a chelating agent that inhibits the degradation of one or more components of the composition. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g., disodium edetate) and citric acid in the weight range of about 0.01% to 0.20% and more preferably in the range of 0.02% to 0.10% by weight by total weight of the composition. The chelating agent is useful for chelating metal ions in the composition that may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Pharmaceutical compositions that are useful in the methods of the invention may be prepared, packaged, or sold in formulations suitable for ophthalmic, oral, rectal, vaginal, parenteral, topical, pulmonary, intranasal, buccal, intravenous, intracerebroventricular, intradermal, intramuscular, or another route of administration. Other contemplated formulations include projected nanoparticles, liposomal preparations, resealed erythrocytes containing the active ingredient, and immunogenic-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

In exemplary embodiments, a pharmaceutical composition comprises a pharmaceutically acceptable excipient, such as a pharmaceutically acceptable carrier, and an exemplary compound described herein.

In certain exemplary embodiments, the pharmaceutical composition comprises, or is in the form of, a pharmaceutically acceptable salt, as generally described below.

The exemplary compounds can be administered in the form of prodrugs. A prodrug can include a covalently bonded carrier which releases the active parent drug when administered to a mammalian subject. Prodrugs can be prepared by modifying functional groups present in the compounds in such a way that the modifications are cleaved, either in routine manipulation or in vivo, to the parent compounds. Prodrugs include, for example, compounds wherein a hydroxyl group is bonded to any group that, when administered to a subject, cleaves to form a free hydroxyl group.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Controlled- or sustained-release formulations of a pharmaceutical composition of the invention may be made using conventional technology.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non toxic parenterally acceptable diluent or solvent, such as water or 1,3 butane diol, for example. Other acceptable diluents and solvents include, but are not limited to, Ringer's solution, isotonic sodium chloride solution, and fixed oils such as synthetic mono or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen free water) prior to parenteral administration of the reconstituted composition.

Transdermal formulations can also be prepared in the form of creams, ointments, salves, sprays, gels, lotions, emulsions, and transdermal patches. Such compositions may contain one or more chemical penetration enhancers, membrane permeability agents, membrane transport agents, emollients, surfactants, stabilizers, and combination thereof.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non toxic parenterally acceptable diluent or solvent, such as water or 1,3 butane diol, for example. Other acceptable diluents and solvents include, but are not limited to, Ringer's solution, isotonic sodium chloride solution, and fixed oils such as synthetic mono or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

Liquid pharmaceutically administrable compositions can, for example, be prepared by dissolving, dispersing, etc., a composition as described herein and optional pharmaceutical adjuvants in an excipient, such as, for example, water, saline aqueous dextrose, glycerol, ethanol, and the like, to thereby form a solution or suspension. If desired, the pharmaceutical composition to be administered can also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, etc. Actual methods of preparing such dosage forms are known, or will be apparent, to those skilled in this art; for example, see Remington's Pharmaceutical Sciences, referenced above.

Liquid suspensions may be prepared using conventional methods to achieve suspension of the HMW-HA or other composition of the invention in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

Dry powder formulations (“DPFs”) with large particle size have improved flowability characteristics, such as less aggregation, easier aerosolization, and potentially less phagocytosis. Dry powder aerosols for inhalation therapy are generally produced with mean diameters primarily in the range of less than 5 microns, although a preferred range is between one and ten microns in aerodynamic diameter. Large “carrier” particles (containing no drug) have been co-delivered with therapeutic aerosols to aid in achieving efficient aerosolization among other possible benefits.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

The regimen of administration may affect what constitutes an effective amount. The therapeutic formulations may be administered to the subject either prior to or after a diagnosis of disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a subject, preferably a mammal, more preferably a human, may be carried out using known procedures, at dosages and for periods of time effective to prevent or treat disease. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

The compound may be administered to a subject as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of the present invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

In one embodiment, the compositions of the invention are administered to the subject in dosages that range from one to five times per day or more.

In another embodiment, the compositions of the invention are administered to the subject in range of dosages that include, but are not limited to, once every day, every two, days, every three days to once a week, and once every two weeks. In another embodiment, the compositions of the invention are administered to the subject in range of dosages applied in sustained release formulations that include, but are not limited to, once every two days, every three days to once a week, and once every two weeks, depending on the precise sustained formulation used and with the goal of maintaining serum concentrations of the therapeutic agents that are therapeutically effective without inducing unacceptable side effects.

It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any subject will be determined by the attending physical taking all other factors about the subject into account.

Compounds of the invention for administration may be in the range of from about 1 mg to about 10,000 mg, about 20 mg to about 9,500 mg, about 40 mg to about 9,000 mg, about 75 mg to about 8,500 mg, about 150 mg to about 7,500 mg, about 200 mg to about 7,000 mg, about 3050 mg to about 6,000 mg, about 500 mg to about 5,000 mg, about 750 mg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments there between.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., a drug used for treating the same or another disease as that treated by the compositions of the invention) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present invention is directed to a packaged pharmaceutical composition comprising a container holding a therapeutically effective amount of a compound or conjugate of the invention, alone or in combination with a second pharmaceutical agent; and instructions for using the compound or conjugate to treat, prevent, or reduce one or more symptoms of a disease in a subject according to the methods of the present invention.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating or preventing a disease in a subject, or delivering an imaging or diagnostic agent to a subject.

Routes of administration of any of the compositions of the invention include oral, nasal, rectal, parenteral, sublingual, transdermal, transmucosal (e.g., sublingual, lingual, (trans) buccal, (trans) urethral, vaginal (e.g., trans- and perivaginally), (intra) nasal, and (trans) rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

In some examples, the composition can be in the form of beads, films, or some other shape, as would be understood by a person of ordinary skill in the art. The size of each individual bead, film, or other shape is of a suitable size for implantation or other form of administration, as would be understood by a person of ordinary skill in the art. Further, the size of each individual bead, film, or other shape may be substantially consistent, or there may be a distribution of different sizes of the respective shape. Optionally, the beads can be implanted, ingested, or otherwise placed inside the body in some way, such that the agent is administered locally or systemically in a sustained-release manner.

It will be appreciated that the composition of the invention may be administered to a subject either alone, or in conjunction with another agent.

In certain embodiments, administration of a composition of the present invention may be performed by single administration or boosted by multiple administrations.

In one embodiment, the invention includes a method comprising administering a combination of compounds described herein. In certain embodiments, the combination has an additive effect, wherein the overall effect of the administering the combination is approximately equal to the sum of the effects of administering each compound. In other embodiments, the combination has a synergistic effect, wherein the overall effect of administering the combination is greater than the sum of the effects of administering each compound.

Optionally, the composition may comprise formulation suitable for delivering the treatment in sustained release formulation capable of releasing the therapeutic substance over period of time period lasting from several hours to several weeks. Such sustained release formulations may consist of osmotic pumps, a fibrin glue, a biocompatible polymer or hydrogel or other means of delivering treatment in a formulation that enables sustained release. The compound can be encapsulated in the polymer or hydrogel such that the agent is slowly released in the body to at least one portion of the tissue or tissues damaged by the pathogen or another inducer of immune exhaustion and/or excessive immune activation. Optionally, the compound can be dispersed throughout the polymer or hydrogel in such a manner to result in slow, sustained release as the polymer or hydrogel degrades inside the body. In some examples, the composition can comprise a biodegradable biocompatible polymer such as polyglycolide or polyglycolic acid (PGA), polylactide or polylactic acid (PLA), poly-L-lactic acid (PLLA), poly-D/L-lactic acid with polyglycolic acid (PDLLA-co-PGA), poly-L-lactic acid-co-glycolic acid (PLGA), PDLLA with bioactive glass, PLGA with bioactive glass, poly-L-lactic acid with β-tricalcium phosphate (PLLA-TCP), poly-L-lactic acid with hydroxyapatite (PLLAHA), polydioxanone (PDS), polyethylene glycol (PEG), poly(8-caprolactone) (PCL), polycaprolactone (PCL) with alginate, polyhydroxybutyrate (PHB), polycarbonate (PC), N-vinyl pyrrolidone copolymers, polyorthoester, chitosan, poly(2-hydroxyethyl-methacrylate) (PHEMA), hyaluronic acid and hydrogels.

These methods described herein are by no means all-inclusive, and further methods to suit the specific application will be apparent to the ordinary skilled artisan. Moreover, the effective amount of the compositions can be further approximated through analogy to compounds known to exert the desired effect.

### Embodiments

Embodiment 1 is a method of preventing death of a subject caused by an exposure to an infectious pathogen, wherein the method comprises an administration of a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof to the subject; and wherein the infectious pathogen is capable of causing a severe or critical disruption or dysfunction in a normal tissue function.

Embodiment 2 is the method of embodiment 1, wherein the infectious pathogen is capable of causing death within about 6 weeks after infecting the subject.

Embodiment 3 is the method of embodiment 1, wherein the administration of the therapeutically effective amount of the pharmaceutical composition is transient and continued for 6 weeks or less.

Embodiment 4 is the method of embodiment 1, wherein the administration of the therapeutically effective amount of the pharmaceutical composition is initiated before the infectious pathogen causes the severe or critical disruption or dysfunction in the normal tissue function in the subject.

Embodiment 5 is a method of preventing, alleviating, or treating a tissue damage, tissue dysfunction, or any combination thereof in a subject in need thereof, wherein the method comprises administering to the subject a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof; and wherein the tissue damage, tissue dysfunction, or any combination thereof is caused by an exposure to an infectious pathogen capable of causing a severe or critical disruption in a normal tissue function.

Embodiment 6 is the method of embodiment 5, wherein the method reduces or reverses the tissue damage, tissue dysfunction, or any combination thereof in the subject.

Embodiment 7 is the method of embodiment 5 or 6, wherein the tissue damage is a damage of a motor function, sensory function, cognitive function, visual function, auditory function, kidney function, respiratory function, hematopoietic system function, normal skin function, salivary gland function, liver function, gall bladder function, gastrointestinal (GI) function, sexual function, or any combination thereof.

Embodiment 8 is the method of any one of embodiments 5-7, wherein the tissue damage is a kidney tissue damage, liver tissue damage, heart tissue damage, lung tissue damage, brain tissue damage, central nervous system damage, peripheral nerve tissue damage, peripheral neuropathy, nephropathy, neutropenia, gastrointestinal tract tissue damage, gut tissue damage, visual system tissue damage, auditory system tissue damage, skin tissue damage, bladder tissue damage, reproductive system tissue damage, hematopoietic system tissue damage, immune system tissue damage, or any combination thereof.

Embodiment 9 is a method of preventing, alleviating, or treating a mitochondrial damage, mitochondrial dysfunction, lysosomal damage, lysosomal dysfunction, or any combination thereof in a subject in need thereof, wherein the method comprises administering to the subject a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof, and wherein the mitochondrial damage, mitochondrial dysfunction, lysosomal damage, lysosomal dysfunction, or any combination thereof is caused by an exposure to an infectious pathogen capable of causing a severe or critical disruption in a normal tissue function.

Embodiment 10 is a method of preventing, alleviating, or treating an axonal damage or dysfunction in a subject in need thereof, wherein the method comprises administering to the subject a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof; and wherein the axonal damage or dysfunction is caused by an exposure to an infectious pathogen capable of causing a severe or critical disruption in a normal tissue function.

Embodiment 11 is a method of preventing, alleviating, or treating at least one gait abnormality in a subject in need thereof, wherein the method comprises administering to the subject a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof; and wherein the at least one gait abnormality is caused by an exposure to an infectious pathogen capable of causing a severe or critical disruption or dysfunction in a normal tissue function.

Embodiment 12 is the method of any one of embodiments 1-11, wherein the infectious pathogen is a viral pathogen, bacterial pathogen, fungal pathogen, or any combination thereof.

Embodiment 13 is the method of any one of embodiments 1-11, wherein the infectious pathogen is a viral pathogen.

Embodiment 14 is the method of embodiment 13, wherein the viral pathogen is a respiratory virus, non-respiratory virus, or a combination thereof.

Embodiment 15 is the method of embodiment 14, wherein the viral pathogen is a respiratory virus.

Embodiment 16 is the method of any one of embodiments 1-11, wherein the infectious pathogen is a bacterial pathogen.

Embodiment 17 is the method of any one of embodiments 1-11, wherein the infectious pathogen is a bacterial respiratory pathogen, bacterial non-respiratory pathogen, or a combination thereof.

Embodiment 18 is the method of any one of embodiments 1-11, wherein the infectious pathogen is a fungal pathogen.

Embodiment 19 is the method of any one of embodiments 1-11, wherein the infectious pathogen is an influenza virus, avian influenza virus, respiratory syncytia virus, coronavirus, Covid-19 virus, severe acute respiratory syndrome (SARS) virus, middle east respiratory syndrome (MERS) virus, retrovirus, parainfluenza virus, adenovirus, metapneumovirus, marburg virus, Ebola virus, hantaviruse, lassa virus, junin virus, Crimea-Congo fever virus, macjupo virus, kyasanur forest virus, dengue fever virus, rabbit fever virus, tularemia virus, rotavirus, cholera pathogen, plague pathogen, pathogen causing toxic shock syndrome, pathogen causing meningococcal meningitis, Staphylococcus aureus (including MRSA), pathogen causing vibrio illness, Hendra virus, Nipah virus, Shigella toxin-producing E. coli, yellow fever pathogen, rocky mountain spotted fever pathogen, diphtheria pathogen, pneumococcal pneumonia pathogen, legionnaire's disease pathogen, Streptococcus spp., Staphylococcus spp., Salmonella spp., Pseudomonas spp., Clostridium spp., Vibrio spp., Mycobacterium spp, or Haemophilus spp., or any combination thereof.

Embodiment 20 is a method of preventing death in a subject in need thereof, wherein the method comprises administering to the subject a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof; and the death is preceded by a hyper-activation of the immune system, hypercytokinemia, cytokine storm, cytokine release syndrome, immune system exhaustion, or any combination thereof caused by a graft-versus-host disease or an exposure to an infectious pathogen capable of causing a severe or critical disruption in a normal tissue function.

Embodiment 21 is the method of embodiment 20, wherein the hyper-activation of the immune system, hypercytokinemia, cytokine storm, cytokine release syndrome, immune system exhaustion, or any combination thereof is caused by a graft-versus-host disease or an exposure to an infectious pathogen capable of causing a severe or critical disruption in a normal tissue function.

Embodiment 22 is the method of embodiments 1-21, wherein the subject has a compromised immune system function.

Embodiment 23 is the method of embodiment 22, wherein the subject has a compromised immune system function due to a genetic mutation, cancer treatment, aging, Alzheimer's disease, sepsis, stress, exposure to radiation, prior or concomitant pathogen infection, graft-versus-host disease, or any combination thereof.

Embodiment 24 is the method of embodiment 23, wherein the subject has an infection caused by an infectious pathogen, was exposed to an infectious pathogen, or a combination thereof, wherein the infectious pathogen is capable of causing a severe or critical disruption in a normal tissue function, wherein a normal tissue function required for survival is reduced by an exposure to the infectious pathogen.

Embodiment 25 is the method of any one of embodiments 22-24, wherein the compromised immune system function comprises a hyper-activation of the immune system, hypercytokinemia, cytokine storm, cytokine release syndrome, immune system exhaustion, or any combination thereof.

Embodiment 26 is a method of preventing, reducing, or reversing a tissue damage, tissue dysfunction, or any combination thereof in a subject in need thereof, wherein the method comprises administering to the subject a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof; and the tissue damage, tissue dysfunction, or any combination thereof is caused by a hyper-activation of the immune system, hypercytokinemia, cytokine storm, cytokine release syndrome, immune system exhaustion, or any combination thereof caused by a graft-versus-host disease, exposure to radiation, exposure to an infectious pathogen, sepsis, or any combination thereof, wherein the infectious pathogen is capable of causing a severe or critical disruption in a normal tissue function.

Embodiment 27 is a method of restoring, improving, or enhancing at least a portion of a tissue function in a subject in need thereof, wherein the method comprises administering to the subject a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof; and the tissue function was reduced by an exposure to an infectious pathogen capable of causing a severe or critical disruption in a normal tissue function, graft-versus-host disease, exposure to radiation, exposure to an infectious pathogen, sepsis, or any combination thereof.

Embodiment 28 is the method of embodiment 27, wherein the subject developed a hyper-activation of the immune system, hypercytokinemia, cytokine storm, cytokine release syndrome, immune system exhaustion, or any combination thereof caused by the exposure to the infectious pathogen.

Embodiment 29 is the method of any one of embodiments 26-28, wherein the tissue function is a motor function, sensory function, cognitive function, visual function, auditory function, kidney function, respiratory function, hematopoietic system function, normal skin function, salivary gland function, liver function, gall bladder function, gastrointestinal (GI) function, sexual function, or any combination thereof.

Embodiment 30 is a method of preventing, alleviating, or treating a mitochondrial damage, mitochondrial dysfunction, lysosomal damage, lysosomal dysfunction, or any combination thereof in a subject in need thereof, wherein the method comprises administering to the subject a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof; and wherein the mitochondrial damage, mitochondrial dysfunction, lysosomal damage, lysosomal dysfunction, or any combination thereof is caused by an exposure to an insult capable of compromising an immune system function, infectious pathogen capable of causing a severe or critical disruption in a normal tissue function, or any combination thereof.

Embodiment 31 is a method of preventing, alleviating, or treating an axonal damage or axonal dysfunction in a subject in need thereof, wherein the method comprises administering to the subject a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof; and wherein the axonal damage or axonal dysfunction is caused by an exposure to an insult capable of compromising an immune system function, infectious pathogen capable of causing a severe or critical disruption in a normal tissue function, or any combination thereof.

Embodiment 32 is a method of preventing, alleviating, or treating at least one gait abnormality in a subject in need thereof, wherein the method comprises administering to the subject a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof; and wherein the at least one gait abnormality is caused by an exposure to an insult capable of compromising an immune system function, infectious pathogen capable of causing a severe or critical disruption in a normal tissue function, or any combination thereof.

Embodiment 33 is the method of any one of embodiments 30-32, wherein the subject has a graft-versus-host disease.

Embodiment 34 is the method of any one of embodiments 30-33, wherein the subject has a compromised immune system due to a graft-versus-host disease.

Embodiment 35 is a method of preventing, alleviating, or treating a hyper-activation of the immune system, hypercytokinemia, cytokine storm, cytokine release syndrome, immune system exhaustion, or any combination thereof in a subject in need thereof, wherein the method comprises administering to the subject a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof, and the hyper-activation of the immune system, hypercytokinemia, cytokine storm, cytokine release syndrome, immune system exhaustion, or any combination thereof is caused by a graft-versus-host disease or an exposure to an infectious pathogen capable of causing a severe or critical disruption in a normal tissue function.

Embodiment 36 is a method of preventing, alleviating, or treating the morbidity or mortality caused by a graft-versus-host disease in a subject in need thereof, wherein the method comprises administering to the subject a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof.

Embodiment 37 is a method of preventing, alleviating, or treating the morbidity or mortality caused by a sepsis in a subject in need thereof, wherein the method comprises administering to the subject a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof; and wherein the sepsis is caused by exposure to an infectious pathogen.

Embodiment 38 is the method of any one of embodiment 1-37, wherein the subject has at least one mutation compromising a function of the subject's immune system.

Embodiment 39 is the method of embodiments 1-38, wherein the subject has ataxia.

Embodiment 40 is the method of embodiment 39, wherein the ataxia is a heritable ataxia.

Embodiment 41 is the method of embodiment 39, wherein the ataxia is an ataxia telangiectasia or other heritable ataxia.

Embodiment 42 is the method of any one of embodiment 1-41, wherein the at least one potassium channel blocker comprises 4-aminopyridine, a derivative of 4-aminopyridine, or a combination thereof.

Embodiment 43 is the method of embodiment 42, wherein the derivative of 4-aminopyridine is a compound comprising the structure of Formula (I)

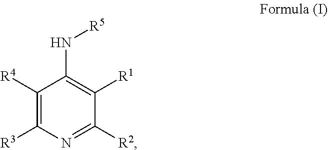

or an analog, a racemate, a tautomer, an isomer, an enantiomer, a diastereomer, a prodrug, or a pharmaceutically acceptable salt thereof; wherein R¹, R², R³, R⁴, and R³ are each independently selected from hydrogen, halogen, C₁-C₆ alkyl, amine, hydroxyl, alkoxy, carboxyl, methyl, methoxy, trifluoromethyl, or any combination thereof; and wherein R¹, R², R³, R⁴, and R⁵ are optionally substituted.

Embodiment 44 is the method of any one of embodiment 1-43, wherein the therapeutically effective amount of the pharmaceutical composition is administered to the subject at the time when exposure to an infectious pathogen capable of causing severe or critical disruptions in normal tissue function, exposure to radiation, or cell transplantation that could cause graft-versus-host disease, first occurs.

Embodiment 45 is the method of any one of embodiment 1-43, wherein the therapeutically effective amount of the pharmaceutical composition is administered to the subject after the time when exposure to an infectious pathogen capable of causing severe or critical disruptions in normal tissue function, exposure to radiation, or cell transplantation that could cause graft-versus-host disease, first occurs.

Embodiment 46 is the method of any one of embodiment 1-43, wherein the therapeutically effective amount of the pharmaceutical composition is administered to the subject after the time when exposure to an infectious pathogen capable of causing severe or critical disruptions in normal tissue function, exposure to radiation, or cell transplantation that could cause graft-versus-host disease, first occurs but before the time when severe or critical changes in normal tissue function are apparent.

Embodiment 47 is the method of any one of embodiment 1-43, wherein the therapeutically effective amount of the pharmaceutical composition is administered to the subject after the time when exposure to an infectious pathogen capable of causing severe or critical disruptions in normal tissue function, exposure to radiation, or cell transplantation that could cause graft-versus-host disease, first occurs but at or after the time when severe or critical changes in normal tissue function are apparent.

Embodiment 48 is the method of any one of embodiment 1-47, wherein the therapeutically effective amount of the pharmaceutical composition is repeatedly administered to the subject for between about 1 day to about 100 years.

Embodiment 49 is the method of any one of embodiment 1-47, wherein the therapeutically effective amount of the pharmaceutical composition is administered to the subject systematically, locally, or a combination thereof.

Embodiment 50 is the method of any one of embodiment 1-47, wherein the therapeutically effective amount of the pharmaceutical composition is administered to the subject by an intraperitoneal injection, intravenous injection, intramuscular injection, intrathecal injection, subcutaneous injection, sublingual administration, inhalation, oral administration, transdermal administration, administration to an outer portion of the body in the form of a liquid, administration to an outer portion of the body in the form of a salve, administration to an outer portion of the body in the form of a bandage, or any combination thereof.

Embodiment 51 is the method of any one of embodiment 1-47, wherein the therapeutically effective amount of the pharmaceutical composition is administered to the subject at a dose of between about 1 mg/day to about 1,000 mg/day of the potassium channel blocker.

Embodiment 52 is the method of any one of embodiment 1-47, wherein the therapeutically effective amount of the pharmaceutical composition is administered to the subject at a dose of between about 2.5 mg/day to about 40 mg/day of the potassium channel blocker.

Embodiment 53 is the method of any one of embodiment 1-47, wherein the therapeutically effective amount of the pharmaceutical composition is administered to the subject at a dose of between about 40 mg/day to about 100 mg/day of the potassium channel blocker.

Embodiment 54 is the method of any one of embodiment 1-53, wherein the therapeutically effective amount of the pharmaceutical composition is co-administered with at least one anticonvulsant agent or a composition thereof.

Embodiment 55 is the method of embodiment 54, wherein the at least one anticonvulsant agent is barbiturate, benzodiazepine, bromide, carbamate, carboxamide, fatty acid, fructose or a derivative thereof, γ-aminobutyric acid (GABA) or an analog thereof, hydantoin, oxazolidinedione, proprionate, pyrimidinedione, pyrrolidine, succinimide, sulfonamide, triazine, urea, valproylamide, or any combination thereof.

Embodiment 56 is the method of any one of embodiments 1-55, wherein the method further enhances cell survival, reduces scarring, or any combinations thereof.

Embodiment 57 is the method of any one of embodiments 1-55, wherein the method further enhances a repair or regeneration of endogenous stem cells, enhances a repair or regeneration of transplanted stem cells, enhances a repair or regeneration of progenitor cells, promotes neural cell generation, enhances cell survival, reduces scarring, decreases lesion size, decreases oxidative damage, or any combinations thereof.

Embodiment 58 is a method of identifying a subject responsive to a 4-aminopyridine administration or a derivative thereof to prevent, alleviate, or treat a tissue damage or tissue dysfunction caused by an exposure to an infectious pathogen, graft-versus-host disease, sepsis, or any combination thereof, wherein the infectious pathogen is capable of causing a severe or critical disruption in a normal tissue function, and wherein the method comprises the steps of: a) administering to the subject between 1 to 5 therapeutically effective amounts of a pharmaceutical composition comprising a 4-aminopyridine, derivative of 4-aminopyridine, or a combination thereof; b) evaluating the symptoms of the tissue damage or tissue dysfunction caused by exposure to an infectious pathogen capable of causing severe or critical disruptions in normal tissue function, graft-versus-host disease, sepsis, or any combination thereof in a subject in need thereof; and c) identifying the subject as responsive to 4-aminopyridine administration to prevent, alleviate, or treat the tissue damage or tissue dysfunction caused by the exposure to the infectious pathogen, graft-versus-host disease, sepsis, or any combination thereof when the symptoms of the tissue damage or tissue dysfunction caused by the exposure to the infectious pathogen, graft-versus-host disease, sepsis, or any combination thereof in the subject improved.

Embodiment 59 is the method of embodiment 58, wherein the tissue damage is a multi-tissue damage, multi-organ tissue damage, or any combination thereof.

### EXPERIMENTAL EXAMPLES

The present invention is further described in detail by reference to the following experimental examples. These examples are provided for purposes of illustration only, and are not intended to be limiting unless otherwise specified. Thus, the present invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1:4-Aminopyridine (4-AP) for Preventing Morbidity and Mortality Induced by Pathogen Infection or by Other Inducers of Cytokine Storms

Although 4-AP has been of interest in chronic illnesses and injuries, there is no evidence of any ability of 4-AP to provide benefit in the case of pathogen infection in vivo in respect to infections, or other physiological insults, that are able to cause hyper-activation of the immune system or, conversely, in situations in which exhaustion or suppression of the immune system occurs (as may occur as a follow on from pathogen infection, leading to a failure to prevent or limit pathogen spread in the body.) Nor is there evidence that 4-AP might provide benefit in the case of pathogen infection in vivo in a subject with an immune system that is compromised by genetic mutations, concomitant infections, medical treatments, aging, Alzheimer's disease, stress, radiation, or other exposures that can compromise normal immune system function.

The approach of central interest to the present studies and invention, which is the application of potassium channel blockers, and preferentially 4-AP or a derivative thereof, to the prevention and/or treatment of the consequences of exposure to an infectious pathogen capable of causing severe or critical disruptions in normal tissue function, or to other conditions in which excess immune system activation can cause severe or critical disruptions in normal tissue function, such as graft-versus-host disease, sepsis and/or radiation (e.g., ionizing radiation, lethal radiation, etc.) has not been considered in respect to such problems.

For example, there is little or no information on 4-AP in respect to any possible effects on management of severe-to-lethal pathogen infections, and only some limited indications of possible utility in mild forms of inflammation of any sort.

The ability of 4-AP to reverse the effects of a potentially lethal, or even severe, infection of any sort is unprecedented. The ability of 4-AP to decrease death of the organism due to any insult is also unprecedented. To prevent death from a potentially lethal insult, particularly after a decline has started, is also unprecedented in prior research on 4-AP. The ability of 4-AP to ameliorate development of co-morbidities of a pathogen infection, including, but not limited to, changes in motor function, is also unprecedented. The ability of 4-AP to overcome the effects of a disease that may involve excessive immune activation, which may be associated with a cytokine storm (i.e., hypercytokinemia) or other aspects of excessive immune activation is unprecedented.

Although there are prior indications that 4-AP is of possible interest in specialized areas of immune cell function (with the caveat that these mostly involve in vitro studies at dosages of 4-AP generally 10-1000 times higher than could be used in vivo), there are no demonstrations of effects in an active infection of any sort in which the insult continues and there is a need to not only mitigate the effects of the infection but also to decrease the infection itself.

The novelty, unexpected results of the present studies, and the unmet need are further emphasized by the nature of publications in this general area. It is particularly important to note that there was no shortage of motivation to develop treatments for conditions of interest relevant to the present invention. Indeed, the development of such treatments has been the focus of extensive efforts in academic and industrial laboratories. There has been enormous interest in regulation of excessive immune activation, in pulmonary infections, in suppression of overly severe immune reactions to pathogens, to acute respiratory distress syndrome (ARDS), in influenza and other respiratory diseases including Covid-19, and in sepsis and other situations (e.g., immunotherapy) in which toxicity caused by cytokine storms is problematic.

One of the important challenges in trying to develop treatments for lethal pathogen infections is that the extent of lethality is extremely variable. For example, influenza kills about 15 to 30% of infected individuals. Trying to conduct experiments is very challenging as the actual lethality occurs in a minority of cases. Moreover, what is particularly needed are treatments for those individuals for whom lethality is more likely to occur. These may be people with genetic mutations, other comorbidities, with metabolic problems, with compromised immune function due to a variety of possible factors such as (but not limited to) stress, aging, Alzheimer's disease, exposure to radiation, or any of a large variety of other issues, and/or with potential other infections.

As the present goals are particularly to protect against human-relevant pathogens capable of causing clinically relevant severe or critical changes, and further to be able to provide such protection even in situations in which the infected individual manifests increased susceptibility to the deleterious effects of infection, initial analyses were conducted in such a manner as to test whether these particularly challenging goals can be met.

In order to address the problems of variability and vulnerability, studies initially focused on a genetic mouse model that is particularly vulnerable to infections. This produced a model of an organism with increased vulnerability while at the same time being relevant to the particular disease modeled by this mouse strain, which is the disease of ataxia telangiectasia, a heritable ataxia. Thus, these mice are of interest in respect to the specific disease they model, while providing a more general model for individuals with an increased vulnerability to pathogen infection and/or that may be associated with existing defects in immune system function that are not caused by the pathogen infection targeted by the methods of this invention. These mice are also useful as a model for ataxias that more generally may have their progression and/or initiation enhanced as a consequence of a pathogen infection.

The mice used in these studies are particularly vulnerable to infection due to being null for the ATM gene. These mice are models for a rare genetic disease caused when people inherit two mutant copies of the ATM gene. More specifically, ataxia-telangiectasia (A-T), caused by mutations in the A-T mutated (ATM) gene, is a neurodegenerative disorder affecting ~1 in 40,000-100,000 children. Recurrent respiratory infections are a common and challenging comorbidity, often leading to the development of bronchiectasis in individuals with A-T. Studies on ATM-null mice infected with influenza A virus (IAV; HKx31, H3N2) in two rounds of infection showed that these mice lost more weight than wild type controls. With this virus strain there was no increased mortality. But secondary infections designed to challenge the B cell memory response with homologous infection (HKx31) and the T cell memory response with heterologous infection (PR8, H1N1) caused more weight loss in the ATM null mice. This enhanced morbidity to secondary infections was not due to failure to effectively clear virus during the primary infection. Instead, Atm-null mice developed persistent peribronchial inflammation, and levels of select serum antibodies to hemagglutinin-specific IAV were significantly lower in Atm-null than WT mice.

Prior studies demonstrated that mice lacking the ATM gene show worse outcomes of influenza virus infection and were deficient in recovery. Following infection, these mice developed an abnormal proximal airway epithelium after infection, with deficient expression of secretoglobin family 1 A member 1 (Scgb1a1) protein—an important immunomodulatory protein that protects the lung against a multitude of respiratory challenges.

When compared with WT mice, naive Atm-null mice had increased airway resistance and reduced lung compliance that worsened during infection before returning to naïve levels by 56 days postinfection (dpi). Although Atm-null lungs appeared pathologically normal before infection by histology, they developed an abnormal proximal airway epithelium after infection that contained E-cadherin⁺, Sox2⁺, and Cyp2f2⁺ cells lacking Scgb1a1 protein expression. Patchy and low expression of Scgb1a1 were eventually observed by 56 dpi. Genetic lineage tracing in HKx31-infected mice revealed club cells require Atm to rapidly and efficiently restore Scgb1a1 expression in proximal airways. Since Scgb1a1 is an immunomodulatory protein that protects the lung against a multitude of respiratory challenges, failure to efficiently restore its expression may contribute to the respiratory diseases seen in individuals with ataxia telangiectasia.

Additional studies demonstrated that mice lacking the ATM gene lose more weight than control mice following infection with influenza A virus (IAV; HKx31, H3N2) in two rounds of infection. Secondary infections designed to challenge the B cell memory response with homologous infection (HKx31) and the T cell memory response with heterologous infection (PR8, H1N1) caused more weight loss in the ATM null mice. ATM-null mice developed persistent peri-bronchial inflammation, and levels of select serum antibodies to hemagglutinin-specific IAV were significantly lower in ATM-null than WT mice.

### Example 2: ATM Knockout (KO) Mice were More Susceptible to Lethal Influenza Infections

Initial experiments established that ATM KO mice were more susceptible to lethal effects of influenza virus infection than wild type (WT) mice. WT type (n=14) and ATM KO mice (n=22) mice were infected with influenza virus (x31) at 5-6 months of age. Mice were followed for 14 days.

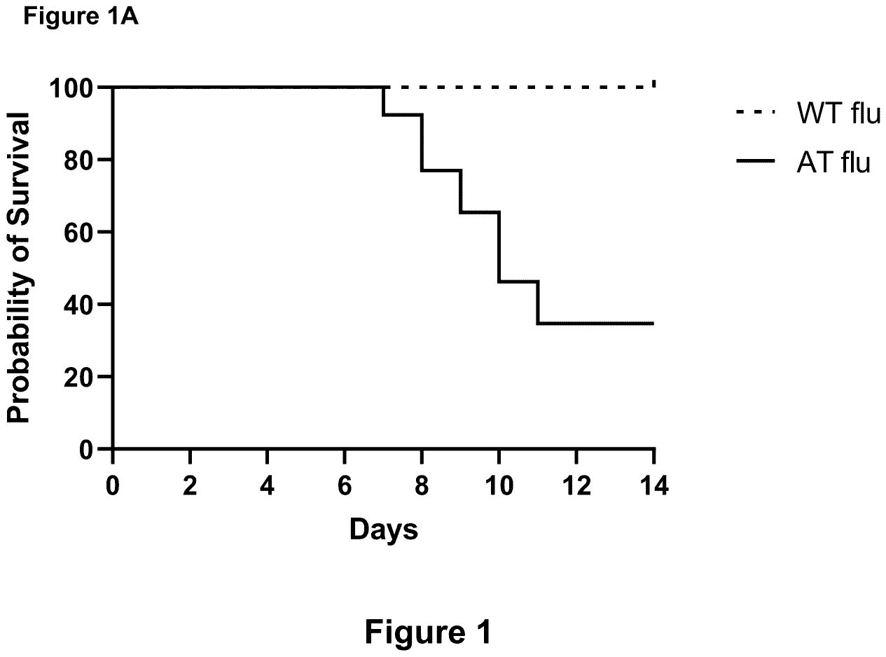

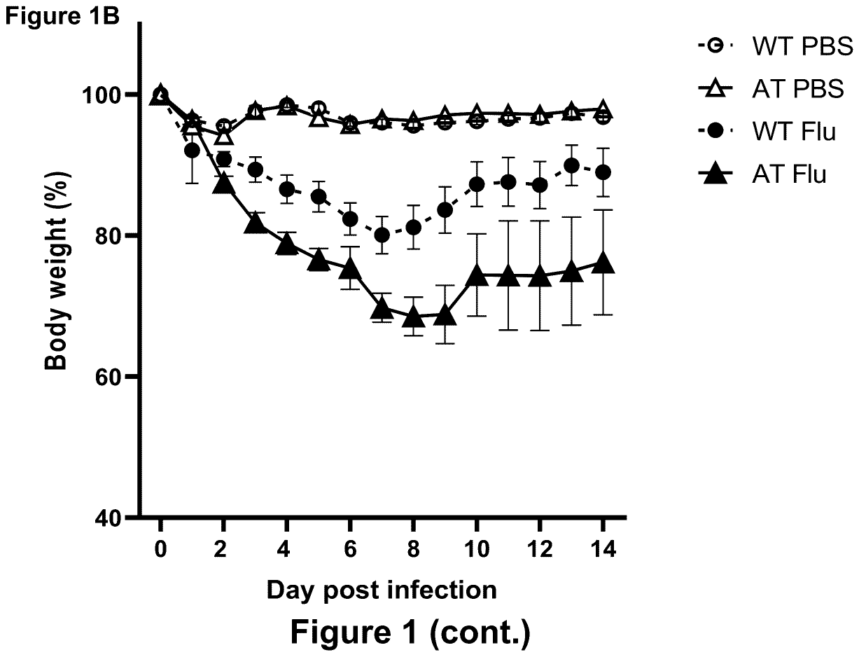

It was observed that, compared to WT, ATM KO mice showed decreased probability of survival (FIG. 1A). Of 14/14 WT mice, 100% survived until at least day 14. In contrast, in ATM KO mice, 18/22 (82%) of the mice died by Day 14, with only 4/22 surviving until this time point.

It was also observed that ATM KO mice showed greater weight loss than WT mice (FIG. 1B). Uninfected mice (dashed lines) showed no weight loss over this time. Infected mice began to lose weight within two days of the infection, but ATM KO mice lost over twice as much weight as WT mice.

Lastly, it was observed that, compared to WT, ATM KO mice infected with influenza virus exhibited morbidity that was noticeable from day 3. Along with the weight loss shown in FIG. 1B, ATM KO mice infected with influenza virus also exhibited lethargy, reduced locomotor activity, ruffled fur, and an unkempt appearance. In addition, motor and coordination deficits were observed in surviving animals.

### Example 3:4-AP Treatment Prevented Influenza-Induced Lethality in ATM KO Animals

In the above studies, it was observed that the first indication of a greater degree of weight loss in ATM KO animals occurred at Day 3. As treatment for a potentially lethal infection will only be initiated when individuals show indications of a worsening health situation, this was the time point at which the tests of this new treatments were initiated.

Having established a greater vulnerability of ATM KO mice to influenza-induced death, the ability of 4-AP to rescue infected animals from death was examined next. In these experiments, 14 male Atm KO mice were infected with influenza virus (x31) at 5-6 months of age. Infected animals were randomly assigned to either treatment with corticosteroid at 1 mg/kg or 4-AP at 1.5 mg/kg for 5 days (3 days post-infection to 7 days post-infection). N=7/treatment group. The studies thus used a low dose of 4-AP that corresponded with −30% of the mouse body surface area equivalent of the dosage of 20 mg/day used in treating multiple sclerosis and was smaller than doses examined in patients with chronic spinal cord injuries.

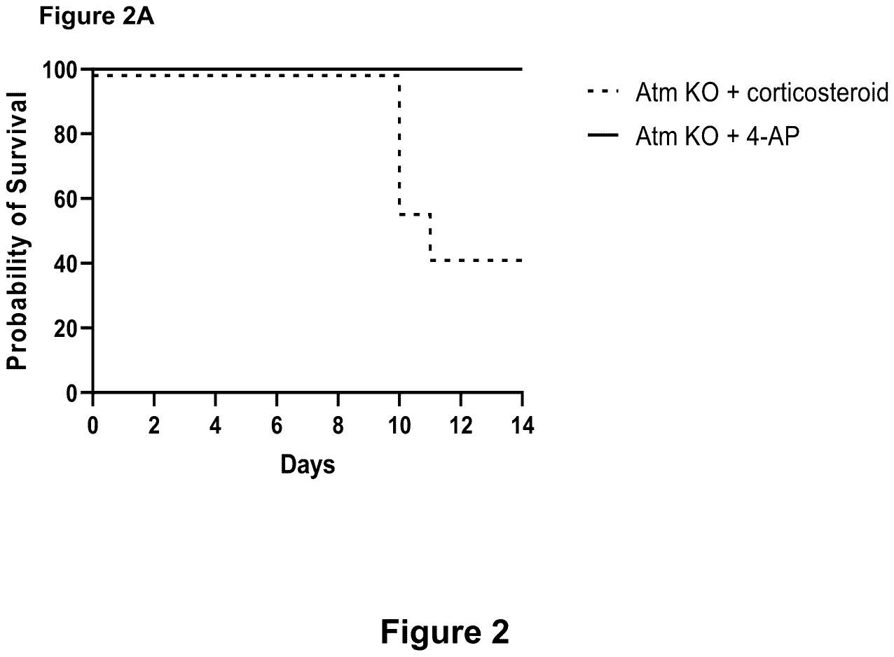

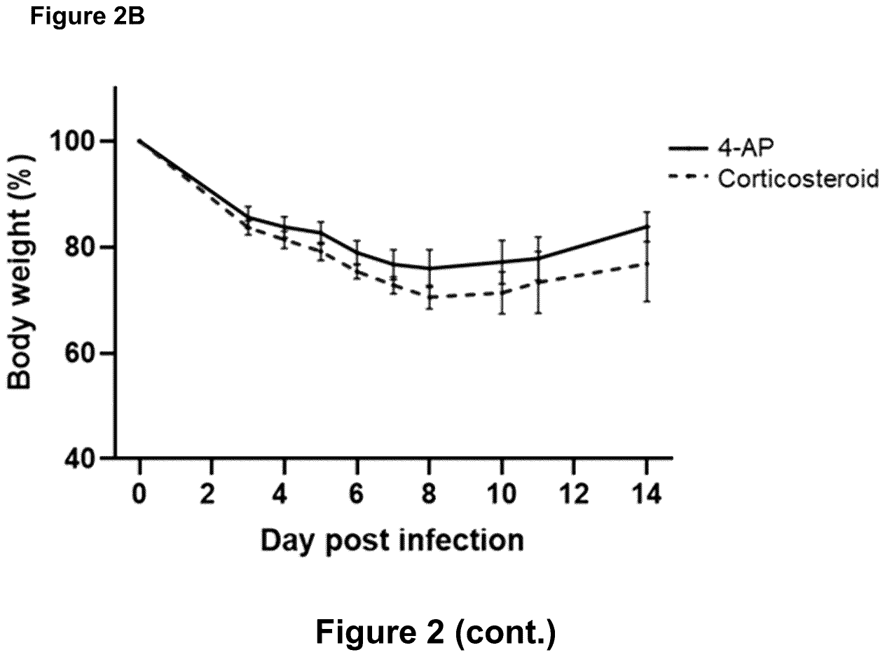

The results showed that all mice (7/7) treated with 4-AP survived until at least Day 14 (FIG. 2A) despite the fact that treatment with 4-AP was only maintained until Day 7 post-infection. In contrast, it was found that treatment with corticosteroids provided mild benefit in terms of survival (FIG. 2A), with 4/7 mice treated with corticosteroids surviving until Day 14 and 3/7 mice dying. Thus, despite corticosteroids being well established anti-inflammatory treatments that are frequently applied in attempts to decrease morbidity or mortality following pathogen infection, this approach was less effective than treatment once daily with 4-AP. This was the case even though the corticosteroids have a somewhat longer predicted half-life in vivo than the 4-AP.

ATM KO mice infected with influenza virus exhibited morbidity that was noticeable from day 3. These changes include weight loss (as shown in FIG. 1B), lethargy and reduced locomotor activity, and ruffled fur and an unkempt appearance. These changes were not modified by treatment with corticosteroid (compare dashed line of FIG. 2B with filled triangles of FIG. 1B), but morbidity was decreased in the 4-AP-treated mice. ATM KO mice infected with influenza and treated with 4-AP showed lesser weight loss, increased activity and motor movement, and healthier coats and a less unkempt appearance.

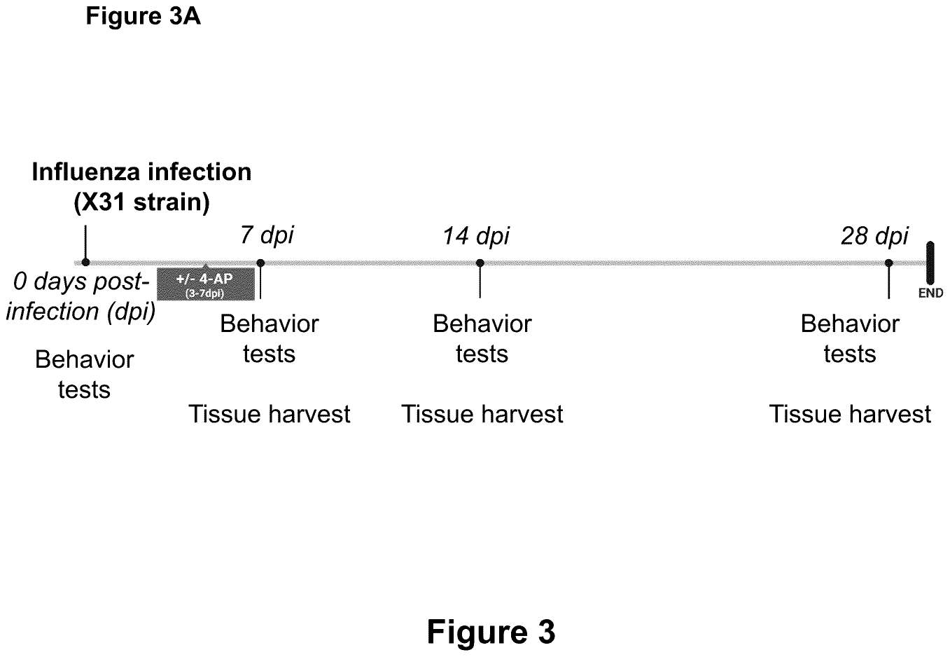

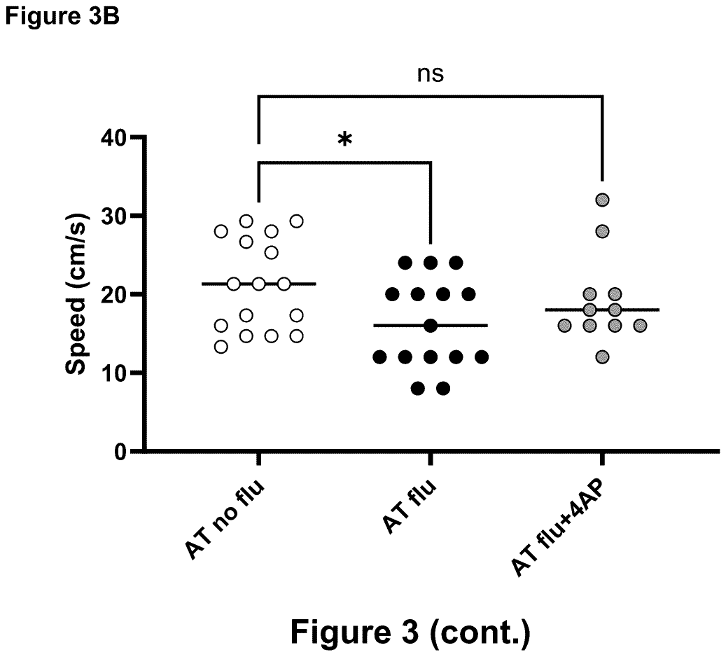

In a separate set of experiments, wildtype and Atm null (AT [M]) animals were exposed (nasally) to a non-neurotropic, non-lethal influenza virus (x31 strain) and were randomly assigned to either treatment with 4-AP (n=11) or saline (n=15) at 1.5 mg/kg for 5 days (3 days post-infection to 7 days post-infection), as shown in FIG. 3A. Mice were pre-trained on the rotarod task.

Mice rapidly became very ill. As in the other experiments described herein, mice showed substantial weight loss by 3 days after infection despite being exposed to a non-lethal infection.

To examine whether 4-AP treatment altered expression of infection-induced motor defects, mice were analyzed using standard rotarod analyses. Animals were exposed to an accelerating rod, three consecutive runs were conducted and the time to fall was determined, as compared with animals prior to infection or animals with no infection at all.

In 54% of saline-treated AT animals, the time to fall between the trial tests significantly decreased (i.e., indicative of impaired motor function). In contrast, in the cohort of AT animals that received 4-AP, only 27% showed a decreased time to fall, indicating a more robust motor function in these mice.

In addition, analysis of the speed at which animals fell off the rotating rod, an indicator of abnormalities in gait, revealed that influenza-infected AT animals fell at a significantly lower speed compared to non-infected AT animals (FIG. 3B). In contrast, the speed that was associated with falling was not significantly decreased in AT infected animals that also received 4-AP. (Two-way Anova p=0.02). Thus, 4-AP treated mice were able to maintain walking function (i.e., gait) required to remain on an accelerating rod.

Taken together, these data indicated that transient 4-AP treatment using a clinically relevant dosage of 4-AP is beneficial in preventing motor function decline in AT animals post infection with a non-lethal influenza strain.

In conclusion, the present studies disclosed a previously unknown and unexpected method to prevent and/or treat damage caused by an infectious pathogen capable of causing severe or critical disruptions in normal tissue function, with the potassium channel blocker 4-AP. The present studies investigated repeatedly administering to an individual exposed to an infectious pathogen capable of causing severe or critical disruptions in normal tissue function, a clinically relevant dosage of 4-AP. This treatment can be initiated at the beginning of or during the progression of the disease. Thus, the present studies provided novel uses of 4-AP in addressing unmet medical needs. The present studies demonstrated unexpected benefits that cannot be predicted from any prior observations.

The present studies also demonstrated a new discovery that 4-AP treatment can be used to treat the effects of exposure to an infectious pathogen capable of causing severe or critical disruptions in normal tissue function. Moreover, the present invention provides a novel treatment of a severe or critical disease condition associated with altered activation of the immune system. The present invention further demonstrated that 4-AP treatment was so effective that it even worked in subjects whose immune function was compromised prior to the infection by insults distinct from those of the pathogen infection.

The disclosures of each and every patent, patent application, and publication cited herein are hereby incorporated herein by reference in their entirety. While the present invention has been disclosed with reference to specific embodiments, it is apparent that other embodiments and variations of the present invention may be devised by others skilled in the art without departing from the true spirit and scope of the invention. The appended claims are intended to be construed to include all such embodiments and equivalent variations.

What is claimed is:

1. A method of preventing, alleviating, or treating a disease, disorder, or condition in a subject in need thereof,
wherein the method comprises administering to the subject a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof;
wherein the disease, disorder, or condition is:
(a) death caused by an exposure to an infectious pathogen capable of causing a severe or critical disruption in a normal tissue function when the method is a method of preventing a condition in the subject;
(b) death preceded by a hyper-activation of the immune system, hypercytokinemia, cytokine storm, cytokine release syndrome, immune system exhaustion, or any combination thereof when the method is a method of preventing a condition in the subject;
(c) a tissue damage, tissue dysfunction, or any combination caused by a hyper-activation of the immune system, hypercytokinemia, cytokine storm, cytokine release syndrome, immune system exhaustion, or any combination thereof caused by a graft-versus-host disease, exposure to radiation, exposure to an infectious pathogen, sepsis, or any combination thereof, wherein the infectious pathogen is capable of causing a severe or critical disruption in a normal tissue function;
(d) a mitochondrial damage, mitochondrial dysfunction, lysosomal damage, lysosomal dysfunction, or any combination thereof caused by an exposure to an insult capable of compromising an immune system function, infectious pathogen capable of causing a severe or critical disruption in a normal tissue function, or any combination thereof;
(e) an axonal damage or axonal dysfunction caused by an exposure to an insult capable of compromising an immune system function, infectious pathogen capable of causing a severe or critical disruption in a normal tissue function, or any combination thereof;
(f) at least one gait abnormality caused by an exposure to an insult capable of compromising an immune system function, infectious pathogen capable of causing a severe or critical disruption in a normal tissue function, or any combination thereof;
(g) a hyper-activation of the immune system, hypercytokinemia, cytokine storm, cytokine release syndrome, immune system exhaustion, or any combination thereof caused by a graft-versus-host disease or an exposure to an infectious pathogen capable of causing a severe or critical disruption in a normal tissue function;
(h) the morbidity or mortality caused by a graft-versus-host disease;
(i) the morbidity or mortality caused by a sepsis, wherein the sepsis is caused by exposure to an infectious pathogen; or
(j) any combination thereof.

2. The method of claim 1, wherein the method is a method of preventing a condition in the subject and the condition is:
(a) death caused by an exposure to an infectious pathogen capable of causing a severe or critical disruption in a normal tissue function;
(b) death preceded by a hyper-activation of the immune system, hypercytokinemia, cytokine storm, cytokine release syndrome, immune system exhaustion, or any combination thereof; or
(c) any combination thereof.

3. The method of claim 2, wherein the hyper-activation of the immune system, hypercytokinemia, cytokine storm, cytokine release syndrome, immune system exhaustion, or any combination thereof is caused by a graft-versus-host disease or an exposure to an infectious pathogen capable of causing a severe or critical disruption in a normal tissue function.

4. The method of claim 2, wherein the infectious pathogen is capable of causing death within about 6 weeks after infecting the subject.

5. The method of claim 2, wherein the administration of the therapeutically effective amount of the pharmaceutical composition is transient and continued for 6 weeks or less.

6. The method of claim 2, wherein the administration of the therapeutically effective amount of the pharmaceutical composition is initiated before the infectious pathogen causes the severe or critical disruption or dysfunction in the normal tissue function in the subject.

7. The method of claim 1, wherein the disease, disorder, or condition is a tissue damage, tissue dysfunction, or any combination caused by a hyper-activation of the immune system, hypercytokinemia, cytokine storm, cytokine release syndrome, immune system exhaustion, or any combination thereof caused by a graft-versus-host disease, exposure to radiation, exposure to an infectious pathogen, sepsis, or any combination thereof,
wherein the infectious pathogen is capable of causing a severe or critical disruption in a normal tissue function.

8. The method of claim 7, wherein the method prevents, reduces, or reverses the tissue damage, tissue dysfunction, or any combination thereof in the subject.

9. The method of claim 7, wherein the tissue damage is a damage of a motor function, sensory function, cognitive function, visual function, auditory function, kidney function, respiratory function, hematopoietic system function, normal skin function, salivary gland function, liver function, gall bladder function, gastrointestinal (GI) function, sexual function, or any combination thereof.

10. The method of claim 7, wherein the tissue damage is a kidney tissue damage, liver tissue damage, heart tissue damage, lung tissue damage, brain tissue damage, central nervous system damage, peripheral nerve tissue damage, peripheral neuropathy, nephropathy, neutropenia, gastrointestinal tract tissue damage, gut tissue damage, visual system tissue damage, auditory system tissue damage, skin tissue damage, bladder tissue damage, reproductive system tissue damage, hematopoietic system tissue damage, immune system tissue damage, or any combination thereof.

12. The method of claim 1, wherein the infectious pathogen is a viral pathogen, bacterial pathogen, fungal pathogen, or any combination thereof.

12. The method of claim 11, wherein the viral pathogen is a respiratory virus, non-respiratory virus, or a combination thereof.

13. The method of claim 1, wherein the infectious pathogen is a respiratory bacterial pathogen, non-respiratory bacterial pathogen, or a combination thereof.

14. The method of claim 1, wherein the infectious pathogen is an influenza virus, avian influenza virus, respiratory syncytia virus, coronavirus, Covid-19 virus, severe acute respiratory syndrome (SARS) virus, middle east respiratory syndrome (MERS) virus, retrovirus, parainfluenza virus, adenovirus, metapneumovirus, marburg virus, Ebola virus, hantaviruse, lassa virus, junin virus, Crimea-Congo fever virus, macjupo virus, kyasanur forest virus, dengue fever virus, rabbit fever virus, tularemia virus, rotavirus, cholera pathogen, plague pathogen, pathogen causing toxic shock syndrome, pathogen causing meningococcal meningitis, Staphylococcus aureus (including MRSA), pathogen causing vibrio illness, Hendra virus, Nipah virus, Shigella toxin-producing E. coli, yellow fever pathogen, rocky mountain spotted fever pathogen, diphtheria pathogen, pneumococcal pneumonia pathogen, legionnaire's disease pathogen, Streptococcus spp., Staphylococcus spp., Salmonella spp., Pseudomonas spp., Clostridium spp., Vibrio spp., Mycobacterium spp, or Haemophilus spp., or any combination thereof.

15. The method of claim 1, wherein the subject has a graft-versus-host disease or ataxia.

16. The method of claim 15, wherein the ataxia is a heritable ataxia.

17. The method of claim 1, wherein the subject has a compromised immune system function.

18. The method of claim 17, wherein the subject has a compromised immune system function due to a genetic mutation, cancer treatment, aging, Alzheimer's disease, sepsis, stress, exposure to radiation, prior or concomitant pathogen infection, graft-versus-host disease, or any combination thereof.

19. The method of claim 17, wherein the subject was exposed to an infectious pathogen or has an infection caused by an infectious pathogen,
wherein the infectious pathogen is capable of causing a severe or critical disruption in a normal tissue function, wherein a normal tissue function required for survival is reduced by an exposure to the infectious pathogen.

20. The method of claim 17, wherein the compromised immune system function comprises a hyper-activation of the immune system, hypercytokinemia, cytokine storm, cytokine release syndrome, immune system exhaustion, or any combination thereof.

21. The method of claim 1, wherein the subject has at least one mutation compromising a function of the subject's immune system.

22. The method of claim 1, wherein the at least one potassium channel blocker comprises 4-aminopyridine, a derivative of 4-aminopyridine, or a combination thereof.

23. The method of claim 22, wherein the derivative of 4-aminopyridine is a compound comprising the structure of Formula (I) 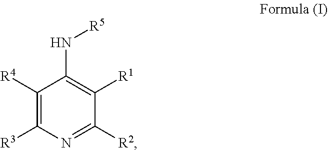
or an analog, a racemate, a tautomer, an isomer, an enantiomer, a diastereomer, a prodrug, or a pharmaceutically acceptable salt thereof,
wherein R¹, R², R³, R⁴, and R⁵ are each independently selected from hydrogen, halogen, C₁-C₆ alkyl, amine, hydroxyl, alkoxy, carboxyl, methyl, methoxy, trifluoromethyl, or any combination thereof; and
wherein R¹, R², R³, R⁴, and R⁵ are optionally substituted.

24. The method of claim 1, wherein the therapeutically effective amount of the pharmaceutical composition is administered to the subject
(a) at the time when exposure to an infectious pathogen capable of causing severe or critical disruptions in normal tissue function, exposure to radiation, or cell transplantation that could cause graft-versus-host disease, first occurs;
(b) after the time when exposure to an infectious pathogen capable of causing severe or critical disruptions in normal tissue function, exposure to radiation, or cell transplantation that could cause graft-versus-host disease, first occurs;
(c) after the time when exposure to an infectious pathogen capable of causing severe or critical disruptions in normal tissue function, exposure to radiation, or cell transplantation that could cause graft-versus-host disease, first occurs but before the time when severe or critical changes in normal tissue function are apparent;
(d) after the time when exposure to an infectious pathogen capable of causing severe or critical disruptions in normal tissue function, exposure to radiation, or cell transplantation that could cause graft-versus-host disease, first occurs but at or after the time when severe or critical changes in normal tissue function are apparent; or
(e) any combination thereof.

25. The method of claim 1, wherein the therapeutically effective amount of the pharmaceutical composition is repeatedly administered to the subject for between about 1 day to about 100 years.

26. The method of claim 1, wherein the therapeutically effective amount of the pharmaceutical composition is administered to the subject systematically, locally, or a combination thereof.

27. The method of claim 1, wherein the therapeutically effective amount of the pharmaceutical composition is administered to the subject by an intraperitoneal injection, intravenous injection, intramuscular injection, intrathecal injection, subcutaneous injection, sublingual administration, inhalation, oral administration, transdermal administration, administration to an outer portion of the body in the form of a liquid, administration to an outer portion of the body in the form of a salve, administration to an outer portion of the body in the form of a bandage, or any combination thereof.

28. The method of claim 1, wherein the therapeutically effective amount of the pharmaceutical composition is administered to the subject at a dose of between about 1 mg/day to about 1,000 mg/day of the potassium channel blocker.

29. The method of claim 28, wherein the therapeutically effective amount of the pharmaceutical composition is administered to the subject at a dose of
(a) between about 2.5 mg/day to about 40 mg/day of the potassium channel blocker; or
(b) between about 40 mg/day to about 100 mg/day of the potassium channel blocker.

30. The method of claim 1, wherein the therapeutically effective amount of the pharmaceutical composition is co-administered with at least one anticonvulsant agent or a composition thereof.

31. The method of claim 30, wherein the at least one anticonvulsant agent is barbiturate, benzodiazepine, bromide, carbamate, carboxamide, fatty acid, fructose or a derivative thereof, γ-aminobutyric acid (GABA) or an analog thereof, hydantoin, oxazolidinedione, proprionate, pyrimidinedione, pyrrolidine, succinimide, sulfonamide, triazine, urea, valproylamide, or any combination thereof.

32. The method of claim 1, wherein the method further enhances cell survival, reduces scarring, or any combinations thereof.

33. The method of claim 1, wherein the method further enhances a repair or regeneration of endogenous stem cells, enhances a repair or regeneration of transplanted stem cells, enhances a repair or regeneration of progenitor cells, promotes neural cell generation, enhances cell survival, reduces scarring, decreases lesion size, decreases oxidative damage, or any combinations thereof.

34. A method of restoring, improving, or enhancing at least a portion of a tissue function in a subject in need thereof,
wherein the method comprises administering to the subject a therapeutically effective amount of a pharmaceutical composition comprising at least one potassium channel blocker or an analog, a racemate, a tautomer, an isomer, an enantiomer, a diastereomer, a prodrug, a pharmaceutically acceptable salt, or a derivative thereof; and
the tissue function was reduced by an exposure to an infectious pathogen capable of causing a severe or critical disruption in a normal tissue function, graft-versus-host disease, exposure to radiation, sepsis, or any combination thereof.

35. The method of claim 34, wherein the subject developed a hyper-activation of the immune system, hypercytokinemia, cytokine storm, cytokine release syndrome, immune system exhaustion, or any combination thereof caused by the exposure to the infectious pathogen.

36. The method of claim 34, wherein the tissue function is a motor function, sensory function, cognitive function, visual function, auditory function, kidney function, respiratory function, hematopoietic system function, normal skin function, salivary gland function, liver function, gall bladder function, gastrointestinal (GI) function, sexual function, or any combination thereof.

37. A method of identifying a subject responsive to an administration of 4-aminopyridine or a derivative thereof to prevent, alleviate, or treat a tissue damage or tissue dysfunction caused by an exposure to an infectious pathogen, graft-versus-host disease, sepsis, or any combination thereof,
wherein the infectious pathogen is capable of causing a severe or critical disruption in a normal tissue function, and
wherein the method comprises the steps of:
a) administering to the subject between 1 to 5 therapeutically effective amounts of a pharmaceutical composition comprising a 4-aminopyridine, a derivative of 4-aminopyridine, or a combination thereof;
b) evaluating the symptoms of the tissue damage or tissue dysfunction caused by exposure to an infectious pathogen capable of causing severe or critical disruptions in normal tissue function, graft-versus-host disease, sepsis, or any combination thereof in a subject in need thereof; and
c) identifying the subject as responsive to 4-aminopyridine administration to prevent, alleviate, or treat the tissue damage or tissue dysfunction caused by the exposure to the infectious pathogen, graft-versus-host disease, sepsis, or any combination thereof when the symptoms of the tissue damage or tissue dysfunction caused by the exposure to the infectious pathogen, graft-versus-host disease, sepsis, or any combination thereof in the subject improved.

38. The method of claim 37, wherein the tissue damage is a multi-tissue damage, multi-organ tissue damage, or any combination thereof.
